(19) United States
(12) Patent Application Publication
(10) Pub. No.: US 2026/0007364 A1
(43) Pub. Date: Jan. 8, 2026

(54) PATIENT MONITORING SENSOR DEVICE AND RELATED METHODS

(71) Applicant: Nonin Medical, Inc., Plymouth, MN (US)

(72) Inventors: Aaron Lobbestael, Plymouth, MN (US); Phil Isaacson, Plymouth, MN (US); Ed Vogel, Plymouth, MN (US); Tim Johnson, Plymouth, MN (US); Justin Beskar, Plymouth, MN (US); Surya Ramachandran, Plymouth, MN (US); Joel Erdman, Plymouth, MN (US); Scott Firman, Plymouth, MN (US); Eric Jaeger, Plymouth, MN (US)

(21) Appl. No.: 19/248,943

(22) Filed: Jun. 25, 2025

Related U.S. Application Data
(60) Provisional application No. 63/667,033, filed on Jul. 2, 2024.

(51) Int. Cl. A61B 5/00; A61B 5/1455

(52) U.S. Cl. CPC A61B 5/6831; A61B 5/14552; A61B 5/6826; A61B 2562/0238; A61B 2562/185

(57) ABSTRACT

A non-invasive patient monitoring sensor configured to monitor at least one physiological parameter of a patient utilizing at least one light emitting LED and at least one photodetector aligned on opposing sides of a sensor site to detect light transmitted thorough a tissue area of the patient. The patient monitoring sensor includes one or more layers of light absorption material configured to enable accurate SpO₂ measurements of patients providing a non-disparate performance for patients of any skin pigmentation type, including light skin pigmentation, intermediate skin pigmentation and dark skin pigmentation. In exemplary aspects, the patient monitoring sensor is configured to have an overall accuracy root mean square (A_{RMS}) of less than 3.0% measured across the saturation range of 70-100% for skin pigmentation types.

### CROSS-REFERENCE TO RELATED APPLICATION

The present application claims the benefit of U.S. Provisional Application No. 63/667,033, filed on Jul. 2, 2024, which is hereby incorporated by reference in its entirety herein.

### TECHNICAL FIELD

The present disclosure is directed to a non-invasive physiological sensor device and related methods of use, in particular a non-invasive pulse oximetry sensor device configured for accurately measuring peripheral oxygen saturation of patients of any skin pigmentation.

### BACKGROUND

Photoplethysmography (PPG) is a ubiquitous technology used in a wide range of clinical and consumer settings with applications in both ambulatory and inpatient settings that involves the non-invasive monitoring of oxygen saturation level in blood-perfused tissue indicative of certain cardiac, pulmonary and vascular conditions. PPG is typically used to measure various blood flow characteristics including, but not limited to, the blood-oxygen saturation of hemoglobin in arterial blood, the volume of individual blood pulsations supplying the tissue, and the rate of blood pulsations corresponding to each heartbeat of a patient. Measurement of these characteristics has been accomplished by use of a non-invasive sensor, which passes light through a portion of the patient's tissue where blood perfuses the tissue, and photoelectrically senses the attenuation of light in such tissue. The amount of light attenuated is then used to calculate the amount of blood constituent being measured. Peripheral oxygen saturation (SpO₂) may be calculated using some form of the classical absorption equation known as Beer's law, which assumes the attenuation is exclusively absorption. The light passed through the tissue is typically selected to be of one or more wavelengths that are absorbed by the blood in an amount representative of the amount of the blood constituent present in the blood. The amount of transmitted light passed through the tissue will vary in accordance with the changing amount of blood constituent in the tissue and the related light absorption. For measuring blood oxygen level, such sensors have been provided with light sources and photodetectors that are adapted to operate at two or more different wavelengths, in accordance with known techniques for measuring blood oxygen saturation. Pulse oximetry is a specification application of PPG that uses two wavelengths of light (typically red and infrared) to determine SpO₂.

Known pulse oximetry sensors based on a two wavelength PPG system used for spot-checking and/or the continuous monitoring of arterial oxygenation can include an optical element which uses one or more light emitting diodes (LEDs) to direct light through blood-perfused tissue, with a photodetector receiving light which has not been absorbed by the tissue. Accurate pulse oximeter measurements require relatively stable positioning of the sensor on an appendage, as well as proper alignment between the light source and light detector.

Accurate measurement of oxygen saturation levels is predicated upon optical sensing in the presence of arterial blood flow. A finger is a convenient body part through which transmitted light will readily pass. Other body parts may also be used, such as other appendages attached to the body (e.g., toes, ears, bridge of nose, etc.). In the case of infants and neonatal babies, the body part may be a foot or hand. Local vascular flow in the body part is dependent on several factors which affect the supply of blood. Blood flow may be affected by centrally mediated vasoconstriction, which must be alleviated by managing the perceived central causes. Peripheral constriction via external compression, however, can be induced by local causes. One such cause of local vasocompression is the pressure exerted by the sensor on the body part.

It is readily recognized that no measurement device is perfectly accurate when used in the real world. For example, two metrics are commonly used for pulse oximeters: bias and accuracy root mean square (A_{RMS}). Both metrics quantify how closely pulse oximeter saturation (SpO₂) readings agree with true oxygen saturation of arterial blood (SaO₂) measured by an arterial blood gas co-oximeter. Mean bias quantifies the average magnitude and direction of the pulse oximeter error. For example, a pulse oximeter with +2% bias systematically overestimates SaO₂ by 2% on average. A device's precision quantifies undirected error, with a higher value of precision indicating more random error. The A_{RMS} metric captures both bias and random error. Mathematically, A_{RMS} is calculated as the square root of the average squared difference between SpO₂ and SaO₂, and it is also related to bias and precision through the square root function of Formula (1):

$${A}_{RMS} = √ ( {bias}^{2} + {precision}^{2} ) \qquad ( 1 )$$

A pulse oximeter with a higher A_{RMS} is less accurate than a pulse oximeter with a lower A_{RMS}. The current standard set by the U.S. Food and Drug Administration (FDA) for cleared devices is an A_{RMS}<3% measured across the saturation range of 70-100% without consideration of skin pigmentation of the clinical population. A_{RMS} is sometimes interpreted analogously to a standard deviation, such that for a pulse oximeter with an A_{RMS} of 3%, a reading of 92% represents a true saturation of ±3% (between 89% and 95%) in 68% of readings, also assuming the mean bias value is zero.

The pulse oximetry measurement depends in part on the assumption that the contribution of light that has not passed through a patient's tissue is negligible. However, there are at least two light sources that do not pass through a patient's tissue causing measurement variations that may result in erroneous blood constituent readings. The first light source that does not pass through a patient's tissue is shunted light, which is light from a sensor's emitter that may be reflected around the exterior of the patient's tissue and sensed by the detector. A second light source that does not pass through a patient's tissue is ambient light, which is external light from an external light source (e.g., light is not from the sensor's emitter) that is sensed by the detector.

Despite the widespread use of pulse oximetry in clinical and consumer settings, the concept that the accuracy of pulse oximetry may be different between people of different skin pigmentations is not new, but the concept garnered increased importance during the Covid-19 pandemic when the discrepancy exhibited a pronounced impact on public health. The skin pigmentation and melanin of a patient may affect a pulse oximetry sensor's ability to accurately measure oxygen saturation, including SpO₂. Pulse oximeters may overestimate blood oxygen levels, especially for people of darker skin pigmentation, potentially causing inaccurate diagnoses and delayed treatment. Research studies have found the overestimation of SpO₂ in patients with dark skin relative to reference SaO₂ measurements obtained by blood gas analysis of extracted blood. Research studies have also concluded that compared to white patients, patients with darker skin pigmentation (e.g., Black, Hispanic, and Asian patients) treated in intensive care units and critically-ill patients with respiratory failure had greater discrepancies between blood saturation levels detected using pulse oximeters versus levels detected in blood samples and received less supplemental oxygen than white patients, resulting in a higher prevalence of occult hypoxemia.

Occult hypoxemia is defined as having a low saturation of arterial blood gas (that is, for example, SaO₂<88%) despite seemingly normal pulse oximetry (that is, for example, SpO₂≥92%). Among critically ill patients, patients with occult hypoxemia detected by arterial blood gas but missed by pulse oximetry have recently been shown to have worse clinical outcomes by detailed analyses—including higher mortality and greater incidence of organ failure—as might be expected given the central role of oxygen delivery in healthy cellular functioning. Clinical action taken by a physician may be dependent upon the conditions and physiological parameters relating to a particular patient. Thus, the overall accuracy of pulse oximeter sensors measuring SpO₂ can be more important at lower measured levels.

The accuracy of arterial oxygen saturation is also a concern in infants whereby a 1.5 fold overestimation of SpO₂ has been observed in darker skin pigmentations than light skin pigmentations. The accuracy of pulse oximeter sensors has also been presented in infants with cyanosis, which can be observed differently in people of different skin pigmentations (e.g., blue/purple in light skin pigmentation, grey/green in intermediate skin pigmentation and grey/white in dark skin pigmentation). A study in 2021 found A_{RMS} of more than 3% in infants overall, with greater discrepancy in darker skin pigmentation (9.5% for Black infants and 8.9% for White infants).

Despite increased policy and regulatory interest and additional research in pulse oximetry technology, the SpO₂—SaO₂ discrepancies have remained a constant issue. This has raised the issue of whether differential accuracy of pulse oximetry due to skin pigmentation may be a contributing factor to health inequality. When a patient has falsely elevated SpO₂ readings from a pulse oximeter, they may be at heightened risk for hidden hypoxemia, which occurs at higher incidence among patients from racial and ethnic minority groups than among the general population, and is associated with higher mortality rates. The accuracy of pulse oximetry differs in subjects of different skin pigmentations to a level that requires particular attention, with many pulse oximeter sensors having decreased accuracy in patients with darker skin pigmentation.

The color and appearance of skin is influenced by several factors, such as the presence of multiple layers with each having its own distinctive optical properties that govern absorption and scattering mechanisms of light, including melanin, keratin, carotene, and hemoglobin, as well as hydration, texture and homogeneity, such that skin is a highly complex organ. While it may be important to distinguish between the characteristics of skin and their optical properties in order to understand the underlying cause of overestimation of SpO₂ readings in patients categorized as having intermediate or darker skin pigmentations, a pulse oximetry sensor that provides accurate SpO₂ readings in patients across light, intermediate and dark skin pigmentations is needed in the industry. This is even more the case in that various standards for categorizing skin color types have been used in the industry, including at least the Fitzpatrick Skin Type Scale, Monk Skin Tone Scale, Individual Typology Angle assessment, and self-reporting.

On Nov. 26, 2023, the FDA published a discussion paper, Approach for Improving the Performance Evaluation of Pulse Oximeter Devices Taking Into Consideration Skin Pigmentation, Race and Ethnicity, which intended to offer an approach to improve the quality of premarket studies and associated methods used to evaluate the performance of pulse oximeters, taking into consideration patient skin pigmentation and patient-reported race and ethnicity. This discussion paper recognized that there is no consensus on the best approach to assess skin pigmentation for medical device development, as neither the current FDA guidance nor the recognized International Organization for Standardization (ISO) standard ISO 80601-2-61 Second Edition 2017-12 (Corrected version 2018-02) for pulse oximeters recommends a particular methodology to assess skin pigmentation. The discussion paper summarized various skin pigmentation assessment methods. The discussion paper also proposed a clinical study design in an effort to reduce the disparate performance of pulse oximeters and thereby increase the overall performance estimates of such devices. As part of the proposed clinical study design, each participant would have the participant's skin pigmentation measured by an instrument at the emitter site of the sensor placement (“sensor site”) and the result categorized by the measurement of Individual Typology Angle (ITA). The objective ITA measurement of sensor site pigmentation is believed to improve consistency in skin pigmentation evaluation leading to a better understanding of any performance differences that may be due to skin pigmentation. The FDA's proposed clinical study design also indicated that it would be appropriate to consider a pulse oximeter to have non-disparate performance when the estimated difference in SpO₂ bias across the ITA and Monk Skin Tone (MST) scale levels is <1.5% when SaO₂>85%, and <3.5% when 70%<SaO₂<85%.

Although there are many questions regarding the accuracy of pulse oximeters in patients with differing skin pigmentation, they are still utilized in a variety of clinical applications and settings. Therefore, as indicated by the FDA's recent discussion papers relating to pulse oximeter devices there is a need in the industry to provide a pulse oximetry sensor device that reduces bias associated with skin pigmentation and is capable of providing accurate measurements irrespective of the skin pigmentation of the patient. There is also a need in the industry to provide a pulse oximetry sensor device that provides accurate measurements for patients of all ages, including adults, pediatrics, infants and neonatal babies, irrespective of their skin pigmentation. There is further a need in the industry for a pulse oximetry sensor device that not only is accurate, efficient, and cost-effective, but that addresses traditional inaccuracies in patients with various skin pigmentations to improve health monitoring outcomes.

### SUMMARY

The present disclosure generally relates to a non-invasive sensor that monitors at least one physiological parameter of a patient utilizing at least one light emitting LED and at least one photodetector, wherein for a transmission type sensor the LED and photodetector are configured to be positioned on opposing sides of a sensor site to detect light transmitted thorough a tissue area of the patient, and wherein for a reflectance transmission type sensor the LED and photodetector are placed on the same side of the sensor site to detect light scattered back to the detector from the tissue area of the patient. In some aspects, the non-invasive sensor is a patient monitoring sensor, which is some preferred aspects is a disposable device. In some other preferred aspects, the non-invasive patient monitoring sensor is a disposable oximeter device.

In one aspect, the present disclosure provides a patient monitoring sensor configured to accurately measure at least one physiological parameter of a patient, such as SpO₂, while providing a non-disparate performance on patients of differing skin pigmentation, including light skin pigmentation, intermediate skin pigmentation and dark skin pigmentation. In exemplary aspects, the patient monitoring sensor is configured to have an overall accuracy root mean square (A_{RMS}) of less than 3.0% measured across the saturation range of 70-100% for patients having light skin pigmentation, intermediate skin pigmentation, and dark skin pigmentation.

In exemplary aspects, each light emitting diode (LED) and each photodetector is contained within an envelope assembly comprising one or more layers of a light absorption material. In some aspects, each light emitting LED and each photodetector is contained within an envelope assembly comprising two layers of a light absorption material. In some aspects, the patient monitoring sensor includes a separate light absorption layer of the light absorption material, which is a separate layer from the one or more layers of the envelope assembly. In some aspects, each light emitting LED and each photodetector is located between at least one layer of the envelope assembly and the light absorption layer. In some preferred aspects, each light emitting LED and each photodetector is contained within the first and second layers of the envelope assembly, such that there is at least one layer of the envelope assembly at least partially located between the light absorption layer and each light emitting LED and each photodetector.

In exemplary aspects, the light absorption material of the envelope assembly and/or the separate light absorption layer includes a light absorption material that is black, or other substantially dark color, provided around each light emitting LED, provided around each photodetector, and provided in the area between each light emitting LED and each photodetector.

In another exemplary aspect, the disclosure provides a patient monitoring system having a patient monitor coupled to a patient monitoring pulse oximetry sensor to measure a physiological parameter. In some preferred aspects, the physiological parameter includes SpO₂.

In another exemplary aspect, the disclosure provides a method of affixing a patient monitoring sensor to a sensor site of a patient to measure a physiological parameter, the sensor site being a tissue area with pulsatile arterial flow, such as a fingertip, toe, earlobe, palm of a hand, or in the case of a neonate across a foot. In some preferred aspects, the light absorption material of the light absorption layer, the first layer of the envelope assembly, the second layer of the envelope assembly, or a combination thereof, is configured to be placed circumferentially around the tissue area of the patient, such as in the case of a fingertip or toe, or at least partially circumferentially around the tissue area of the tissue area of the patient, such as in the case of an earlobe, palm of a hand, or a foot.

In some aspects, the patient monitoring sensor is configured to provide an A_{RMS} value less than 3.0, in some aspects less than 2.5, in some aspects less than 2.4, in some aspects less than 2.3, in some aspects less than 2.2, in some aspects less than 2.1, and in some preferred aspects less than 2.0, measured across the saturation range of 70-100%, measured across the saturation range of 70-80%, in some aspects measured across the saturation range of 80-90%, in some aspects measured across the saturation range of 88-92%, for light skin pigmentation type, intermediate skin pigmentation type, dark skin pigmentation type, or skin pigmentation types.

In some aspects, the patient monitoring sensor is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 70-80%, in some aspects measured across the saturation range of 80-90%, in some aspects measured across the saturation range of 88-92%, for light skin pigmentation type, intermediate skin pigmentation type, dark skin pigmentation type, or a combination of the foregoing skin pigmentation types.

In some aspects, the patient monitoring sensor is configured to generate at least one electrical signal relating to at least one physiological parameter of the patient, the patient monitoring sensor including a conformable substrate body having a first end and an opposing second end, the conformable substrate body having a first surface configured to be located away from the patient when the device is applied to the patient, and the conformable substrate body having a second surface configured to be directed toward a tissue area of the patient when the device is applied to the patient; an envelope assembly comprising a first layer and an opposing second layer, the first layer being configured to be located closer to the second surface of the conformable substrate body than the second layer, the second layer having a first aperture spaced apart a distance from a second aperture, and the envelope assembly having a first end and an opposing second end; and a transmission sensor assembly located between the first and second layers of the envelope assembly, the transmission sensor assembly comprising at least one light emitter located proximate the first aperture and at least one light detector located proximate the second aperture, wherein the at least one light emitter configured to transmit at least a first wavelength and at least a second wavelength, and the at least one light detector configured to detect each of the at least first and second wavelengths, wherein the at least one light emitter and the at least one light detector configured to be positioned on opposing sides of the tissue area of the patient when the device is applied to the patient, such that the light detector is configured to operably engage with the light emitter to detect the at least first and second wavelengths transmitted by the at least one light emitter through at least a portion of the tissue area of the patient, wherein at least the first and second layers of the envelope assembly comprising a light absorption material configured to reduce or prevent the at least one light detector from detecting the at least first wavelength that does not pass through an intended light path through the portion of the tissue area of the patient from the at least one light emitter.

In some aspects, the patient monitoring sensor further includes a light absorption layer comprising a light absorption material configured to reduce or prevent the at least one light detector from detecting the at least first wavelength that does not pass through an intended light path through the portion of the tissue area of the patient from the at least one light emitter, wherein the light absorption layer having a third aperture and a fourth aperture, wherein the envelope assembly is provided between the conformable substrate body and the light absorption layer, such that the first aperture of the envelope assembly and the third aperture of the light absorption layer have a first aligned configuration and the second aperture of the envelope assembly and the fourth aperture of the light absorption layer have a second aligned configuration.

In some aspects, the patient monitoring sensor further includes a light absorption layer comprising a light absorption material provided between the conformable substrate body and the envelope assembly to reduce or prevent the at least one light detector from detecting the at least first wavelength that does not pass through an intended light path through the portion of the tissue area of the patient from the at least one light emitter.

In some aspects, the patient monitoring sensor is configured to generate at least one electrical signal relating to at least one physiological parameter of the patient, the patient monitoring sensor including a conformable substrate body having a first end and an opposing second end, the conformable substrate body having a first surface configured to be located away from the patient when the device is applied to the patient, and the conformable substrate body having a second surface configured to be directed toward a tissue area of the patient when the device is applied to the patient; an envelope assembly comprising at least one layer having a first aperture spaced apart a distance from a second aperture, wherein the envelope assembly having a first end and an opposing second end, a light absorption layer comprising a light absorption material provided to at least partially engage with the second surface of the conformable substrate body, and a transmission sensor assembly located between the at least one layer of the envelope assembly and the light absorption layer, the transmission sensor assembly comprising at least one light emitter located proximate the first aperture and at least one light detector located proximate the second aperture, wherein the at least one light emitter configured to transmit at least a first wavelength and at least a second wavelength, and the at least one light detector configured to detect each of the at least first and second wavelengths, wherein the at least one light emitter and the at least one light detector configured to be positioned on opposing sides of the tissue area of the patient when the device is applied to the patient, such that the light detector is configured to operably engage with the light emitter to detect the at least first and second wavelengths transmitted by the at least one light emitter through at least a portion of the tissue area of the patient, wherein the light absorption layer is configured to reduce or prevent the at least one light detector from detecting the at least first wavelength that does not pass through an intended light path through the portion of the tissue area of the patient from the at least one light emitter.

In some aspects, the envelope assembly comprises a first and an opposing second layer, the first layer configured to be located closer to the second surface of the conformable substrate body and the light absorption layer than the second layer, and wherein the transmission sensor assembly located between the first and second layers of the envelope assembly.

In some aspects, the light absorption layer is provided at least partially between the second surface of the conformable substrate body and the first layer of the envelope assembly.

In some aspects, each of the first and second layers of the envelope assembly also include a light absorption material to reduce or prevent the at least one light detector from detecting the at least first wavelength that does not pass through an intended light path through the tissue area of the patient from the at least one light emitter.

In some aspects, the envelope assembly comprising a single sheet of light absorption material in a folded configuration forming the first and second layers.

In some aspects, the first and second layers of the envelope assembly are formed of separate sheets of the light absorption material.

In some aspects, the light absorption layer includes a third aperture and a fourth aperture, wherein the envelope assembly is provided between the conformable substrate body and the light absorption layer, such that the first aperture of the envelope assembly and the third aperture of the light absorption layer having a first aligned configuration located proximate the at least one light emitter and the second aperture of the envelope assembly and the fourth aperture of the light absorption layer having a second aligned configuration located proximate the at least one light detector.

In some aspects, the light absorption layer has a greater surface area than the at least one layer of the envelope assembly. In some aspects, the light absorption layer has a greater surface area than each of the first and second layers of the envelope assembly.

In some aspects, the light absorption layer having a first pair of opposing tab members located proximate the first end of the conformable substrate body, and the light absorption layer having a second pair of tab members located proximate the second end of the conformable substrate body, wherein an intermediate section of the light absorption layer located between the first and second pair of tabs has a narrower width than each of the first and second pair of tabs.

In some aspects, the conformable substrate body having a first pair of opposing tab members located proximate the first end, the conformable substrate body having a second pair of opposing tab members located proximate the second end, wherein the second pair of opposing tab members has at least one elongated tape member having a length that is greater than the opposing tab member, and wherein an intermediate section of the conformable substrate body located between the first and second ends has a width that is less than each of the first and second ends.

In some aspects, the second surface of the conformable substrate body having a first adhesive layer, wherein at least a portion of the first adhesive layer is configured to be in direct contact with at least portion of the tissue area of the patient when the device is applied to the patient.

In some aspects, the light absorption layer includes a plurality of slits, wherein the plurality of slits are configured to reduce or prevent the at least one light detector from detecting the at least first wavelength that does not pass through an intended light path through the portion of the tissue area of the patient from the at least one light emitter.

In some aspects, the patient monitoring sensor includes a connector in electrical or optical communication with the transmission sensor assembly via at least one lead, wherein the electrical connector is configured to be connected to a monitor, and wherein the monitor is configured to receive the at least one signal generated by the transmission sensor assembly relating the at least one physiological parameter of the patient.

In some aspects, the at least one light emitter is configured to the first wavelength between about 620 nm and about 750 nm and the second wavelength between about 780 nm and about 1200 nm.

In some aspects, the second surface of the conformable substrate body includes a first adhesive layer, wherein at least a portion of the first adhesive layer is configured to be in direct contact with at least portion of the tissue area of the patient when the device is applied to the patient.

In some aspects, at least one surface of the conformable substrate body is configured to be in direct contact with a retention mechanism, and when the device is applied to the patent the retention mechanism maintains the patient monitoring sensor in direct contact with the tissue area of the patient.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 illustrates a perspective view of an exemplary patient monitoring sensor affixed to a finger of a patient, in accordance with certain embodiments.

FIG. 2A illustrates a top perspective view of an exemplary patient monitoring sensor, in accordance with certain embodiments.

FIG. 2B illustrates a top perspective view of an exemplary patient monitoring sensor, in accordance with certain embodiments.

FIG. 2C illustrates a top perspective view of an exemplary patient monitoring sensor, in accordance with certain embodiments.

FIG. 2D illustrates a top perspective view of an exemplary patient monitoring sensor, in accordance with certain embodiments.

FIG. 2E illustrates a top perspective view of an exemplary patient monitoring sensor, in accordance with certain embodiments.

FIG. 3 illustrates a bottom perspective view of an exemplary patient monitoring sensor, in accordance with certain embodiments.

FIG. 4 illustrates an exploded perspective view of an exemplary patient monitoring sensor, in accordance with certain embodiments.

FIG. 5 illustrates an exemplary substrate body of an exemplary patient monitoring sensor, in accordance with certain embodiments.

FIG. 6 illustrates an exemplary construction assembly of a transmission sensor assembly within an envelope assembly, in accordance with certain embodiments.

FIG. 7A illustrates a bottom perspective view of an exemplary patient monitoring sensor, in accordance with certain embodiments.

FIG. 7B illustrates a bottom perspective view of an exemplary patient monitoring sensor, in accordance with certain embodiments.

FIG. 8A illustrates an exploded perspective view of an exemplary patient monitoring sensor, in accordance with certain embodiments.

FIG. 8B illustrates an exploded perspective view of an exemplary patient monitoring sensor, in accordance with certain embodiments.

FIG. 8C illustrates an exploded perspective view of an exemplary patient monitoring sensor, in accordance with certain embodiments.

FIG. 8D illustrates an exploded perspective view of an exemplary patient monitoring sensor, in accordance with certain embodiments.

FIG. 9A illustrates an exemplary layer of light absorption material of an exemplary patient monitoring sensor, in accordance with certain embodiments.

FIG. 9B illustrates an exemplary layer of light absorption material of an exemplary patient monitoring sensor, in accordance with certain embodiments.

FIG. 10 illustrates an exemplary combination of an envelope assembly and an additional layer of light absorption material of an exemplary patient monitoring sensor, in accordance with certain embodiments.

FIG. 11 illustrates a bottom perspective view of an exemplary patient monitoring sensor, in accordance with certain embodiments.

FIG. 12A illustrates an exploded perspective view of an exemplary patient monitoring sensor, in accordance with certain embodiments.

FIG. 12B illustrates an exploded perspective view of an exemplary patient monitoring sensor, in accordance with certain embodiments.

FIG. 13 illustrates an exemplary combination of an envelope assembly and an additional layer of light absorption material of an exemplary patient monitoring sensor, in accordance with certain embodiments.

FIG. 14 illustrates a perspective view of an exemplary patient monitoring sensor affixed to a toe of a patient, in accordance with certain embodiments.

FIG. 15 illustrates a top perspective view of an exemplary patient monitoring sensor, in accordance with certain embodiments.

FIG. 16 illustrates a bottom perspective view of an exemplary patient monitoring sensor, in accordance with certain embodiments.

FIG. 17 illustrates an exploded perspective view of an exemplary patient monitoring sensor, in accordance with certain embodiments.

FIG. 18 illustrates a perspective view of an exemplary patient monitoring sensor affixed to a foot of a patient, in accordance with certain embodiments.

FIG. 19 illustrates a top perspective view of an exemplary patient monitoring sensor, in accordance with certain embodiments.

FIG. 20 illustrates a bottom perspective view of an exemplary patient monitoring sensor, in accordance with certain embodiments.

FIG. 21 illustrates an exploded perspective view of an exemplary patient monitoring sensor, in accordance with certain embodiments.

FIG. 22 illustrates an exemplary construction of a transmission sensor assembly within an envelope assembly, in accordance with certain embodiments.

FIG. 23A illustrates an exemplary layer of light absorption material of an exemplary patient monitoring sensor, in accordance with certain embodiments.

FIG. 23B illustrates an exemplary layer of light absorption material of an exemplary patient monitoring sensor, in accordance with certain embodiments.

FIG. 24A illustrates an exemplary combination of an envelope assembly and an additional extending layer of light absorption material of an exemplary patient monitoring sensor, in accordance with certain embodiments.

FIG. 24B illustrates an exemplary combination of an envelope assembly and an additional extending layer of light absorption material of an exemplary patient monitoring sensor, in accordance with certain embodiments.

FIG. 25 illustrates the steps of applying an exemplary patient monitoring sensor to a finger of a patient, in accordance with certain embodiments.

FIG. 26 illustrates the steps of applying an exemplary patient monitoring sensor to a toe of a patient, in accordance with certain embodiments.

FIG. 27 illustrates the steps of applying an exemplary patient monitoring sensor to a foot of a patient, in accordance with certain embodiments.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The present disclosure is directed to a device, systems using the device, and related methods of using the device, to monitor one or more physiological parameters of a patient. In some preferred aspects, the device is a patient monitoring sensor configured to measure the oxygenation of a patient, particularly SpO₂ measurements of the patient. In some aspects, the device is a patient monitoring sensor configured to include one or more optical emitters, one or more optical detectors, a temperature sensor, a pressure sensor, an accelerometer, or a combination thereof, to provide measured data for a variety of blood and tissue measurements. For example, a patient monitoring sensor and a suitable algorithm can be configured to provide data as to the following physiological parameters or conditions: Regional saturation (rSO2), Hemoglobin (Hb) concentration in tissue, Tissue temperature, SpO₂, Total hemoglobin (tHb), Hematocrit, Anemia, CO₂, COHb, MetHb, pH, Respiration, Perfusion, Apnea, Hypopnea, Pulse wave velocity, Blood pressure, Interstitial pressure, Arterial stiffness, Intracranial Pressure, Intrauterine pressure/contractions, Glucose, Cardiac output, Bilirubin, Hydration, Hematoma, Vascular compliance, Tissue viability, Malaria, Blood cancer, Thrombocytopenia (low platelet count), Sepsis, Thrombosis, Compartment syndrome, Hypoxic burden, Fractional tissue oxygen extraction, or a combination thereof. In some preferred aspects, a PPG waveform obtained by using a pulse oximetry sensor can be used in relation to one or more of the foregoing physiological parameters or conditions.

The present disclosure recognizes problems associated with patient monitoring sensors utilizing LED(s) and photodetector(s) on patients having different skin pigmentation types, including the disparate performance of pulse oximeter sensors. Accordingly, the present disclosure describes a non-invasive pulse oximetry sensor configured to measure SpO₂ of patients of any skin pigmentation type, including light skin pigmentation, intermediate skin pigmentation and dark skin pigmentation. The measure of SpO₂ of patients of various skin pigmentation provides for a mitigation of the overestimation or underestimation of blood oxygen levels in such patients that is an on-going issue in prior art pulse oximeter devices, which can lead to occult hypoxemia. The non-invasive pulse oximetry sensor is also capable of measuring data as to the foregoing parameters or conditions, in some preferred aspects including at least the pulse rate of the patient.

The term “A_{RMS}” used herein refers to the metric for pulse oximeter devices that is calculated by taking the square root of the sum of the square of the bias plus the square of the precision, as shown by Formula (1):

$${A}_{RMS} = √ ( {bias}^{2} + {precision}^{2} ) \qquad ( 1 )$$

whereby bias is the mean difference between SaO₂ and SpO₂ readings, and precision is the standard deviation of the differences between SaO₂ and SpO₂.

The terms “Individual Typology Angle” and “ITA” used herein refer to an integrated objective quantitative measure of constitutive skin pigmentation using a spectrophotometer instrument to calculate an ITA value. In some aspects, the spectrophotometer instrument may be a handheld, portable instrument designed to evaluate the color and appearance of skin samples that may have a generally flat, shaped or curved surface.

The term “ITA value” used herein refers to the calculated value by the spectrophotometer using the equation ITA=180/π×arctan ((L*−50)/b*), whereby standard colorimetry approaches are used to measure Commision Internationale d'Eclairage L*a*b* (CIELAB) calorimetric parameters, where L* is luminance ranging from black (0) to white (100), a* is the red/green component, and b* is the yellow/blue component. An exemplary spectrophotometer being a normally operating Konica Minolta CM-700d.

The term “ITA skin color type” used herein refers to the classification of skin measured using ITA as a metric based upon the resulting ITA value, whereby, for example, skin color has typically been categorized into six (6) categories (very light skin has an ITA value >55°, light skin has an ITA value of 41° to <55°, intermediate skin has an ITA value of 28° to <41°, tan skin has an ITA value of 10° to <28°, brown skin has an ITA value of −30° to <10°, and dark skin has an ITA value <−30°. ITA values are measured at various locations of the body, for example, at the surface directly in contact with the sensor light emitter. For instance, for fingertip sensors, ITA values are measured at the mid-dorsal pigmented skin surface of the distal phalanx, proximal to the eponychium to capture the widest variation in skin pigmentation applicable to sensor placement, as recommended by FDA protocols. Other locations of the body that ITA values may be measured at include, but are limited to, forehead, armpit, foot, hand, and the like.

The term “Fitzpatrick skin color types” as used herein refers to the skin phototype categories based on the subjective Fitzpatrick scale as a way to estimate the response of different types of skin to ultraviolet (UV) light, whereby the Fitzpatrick skin type classification is summarized in Table 1.

TABLE 1
Fitzpatrick skin color type

| Classification | Description |
|---|---|
| Fitzpatrick skin | skin always burns, never tans, and is |
| type I | sensitive to UV exposure |
| Fitzpatrick skin | skin burns easily and tans minimally |
| type II | |
| Fitzpatrick skin | skin burns moderately and tans gradually |
| type III | to light brown |
| Fitzpatrick skin | skin burns minimally and always tans well |
| type IV | to moderately brown |
| Fitzpatrick skin | skin rarely burns and tans profusely to dark |
| type V | |
| Fitzpatrick skin | skin never burns, is deeply pigmented, and is |
| type VI | least sensitive to UV exposure |

The term “Monk Skin Tone Scale” or “MST Scale” as used herein refers to the 10-shade scale developed by Dr. Ellis Monk at Harvard that provides a subjective skin tone scale for human skin, whereby the ten digitally defined colors of the MST Scale as defined in CIELAB (ISO/CIE 11664-4 Colorimetry-Part 4: CIE 1976 L*a*b*) color space is summarized in Table 2.

TABLE 2
Monk Skin Tone Scale

| MST Scale | | | |
|---|---|---|---|
| Level | L* | a* | b* |
| 1 | 94.2 | 1.5 | 5.4 |
| 2 | 92.3 | 2.1 | 7.3 |
| 3 | 93.1 | 0.2 | 14.2 |
| 4 | 87.6 | 0.5 | 17.7 |
| 5 | 77.9 | 3.5 | 23.1 |
| 6 | 55.1 | 7.8 | 26.7 |
| 7 | 42.5 | 12.3 | 20.5 |
| 8 | 30.7 | 11.7 | 13.3 |
| 9 | 21.1 | 2.7 | 6.0 |
| 10 | 14.6 | 1.5 | 3.5 |

The terms “light skin pigmentation” or “light skin pigmentation type” used herein refers to a skin pigmentation classification at the light skin portion of a skin pigmentation spectrum, whereby the skin pigmentation spectrum commonly described in terms of light skin pigmentation, intermediate skin pigmentation, and dark skin pigmentation. In some exemplary classifications, the light skin pigmentation type has a measured ITA value being ≥41°. In some other exemplary classifications, the light skin pigmentation type has a measured ITA value being >30°. In some other exemplary classifications, the light skin pigmentation type is included within the MST Scale levels 1-3. In some other exemplary classifications, the light skin pigmentation type is included within the Fitzpatrick skin types I-II.

The terms “intermediate skin pigmentation” or “intermediate skin pigmentation type” used herein refers to a skin pigmentation classification between light skin pigmentation and dark skin pigmentation on the skin pigmentation spectrum, whereby the skin pigmentation spectrum commonly described in terms of light skin pigmentation, intermediate skin pigmentation, and dark skin pigmentation. In some exemplary classifications, the intermediate skin pigmentation type has a measured ITA value in the range of 41°>ITA>10°. In some other exemplary classifications, the intermediate skin pigmentation type has a measured ITA value being 30°≥ITA≥−30°. In some other exemplary classifications, the intermediate skin pigmentation type is included within the MST Scale levels 4-7. In some other exemplary classifications, the intermediate skin pigmentation type is included within the Fitzpatrick skin types III-IV.

The terms “dark skin pigmentation” or “dark skin pigmentation type” used herein refers to a skin pigmentation classification at the dark skin portion of a skin pigmentation spectrum, whereby the skin pigmentation spectrum commonly described in terms of light skin pigmentation, intermediate skin pigmentation, and dark skin pigmentation. In some exemplary classifications, the dark skin pigmentation type has a measured ITA value being <10°. In some other exemplary classifications, the dark skin pigmentation type has a measured ITA value being <−30°. In some other exemplary classifications, the dark skin pigmentation type is included within the MST Scale levels 8-10. In some other exemplary classifications, the light skin pigmentation type is included within the Fitzpatrick skin types V-VI.

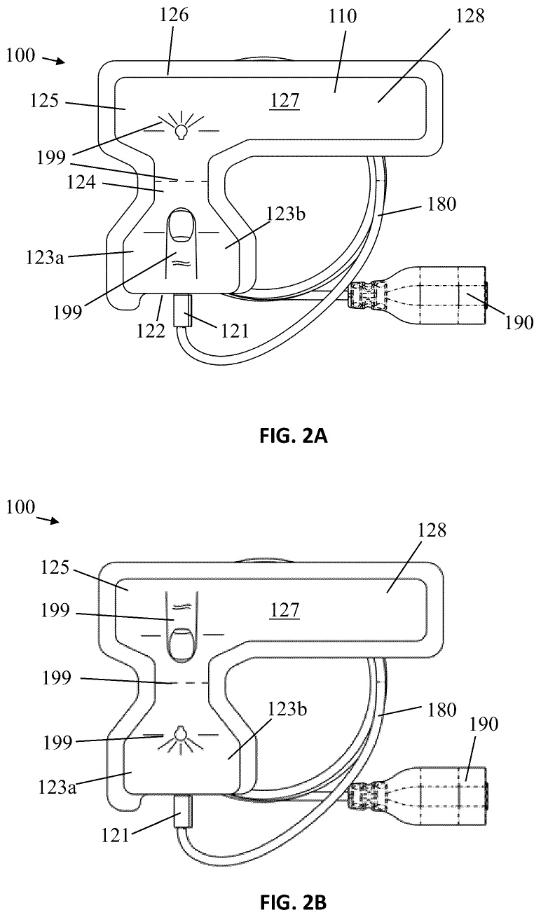

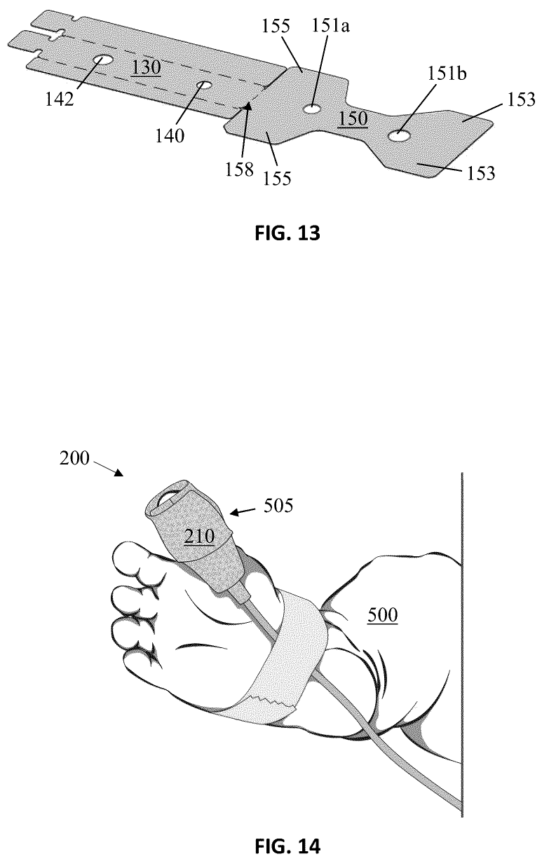

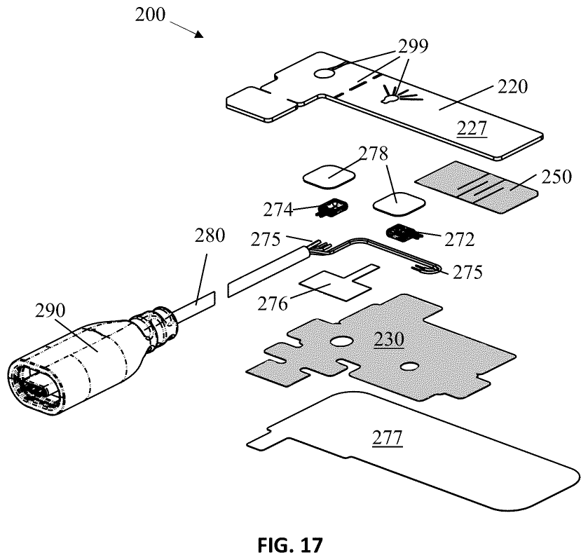

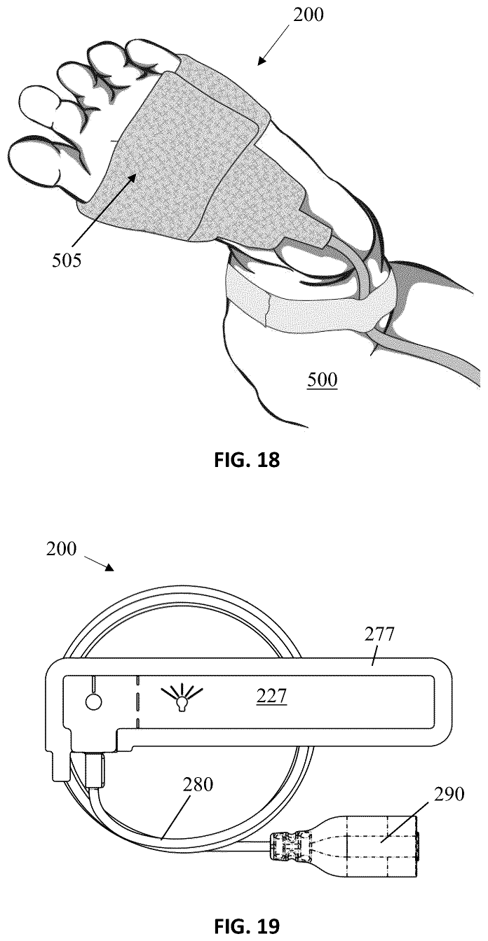

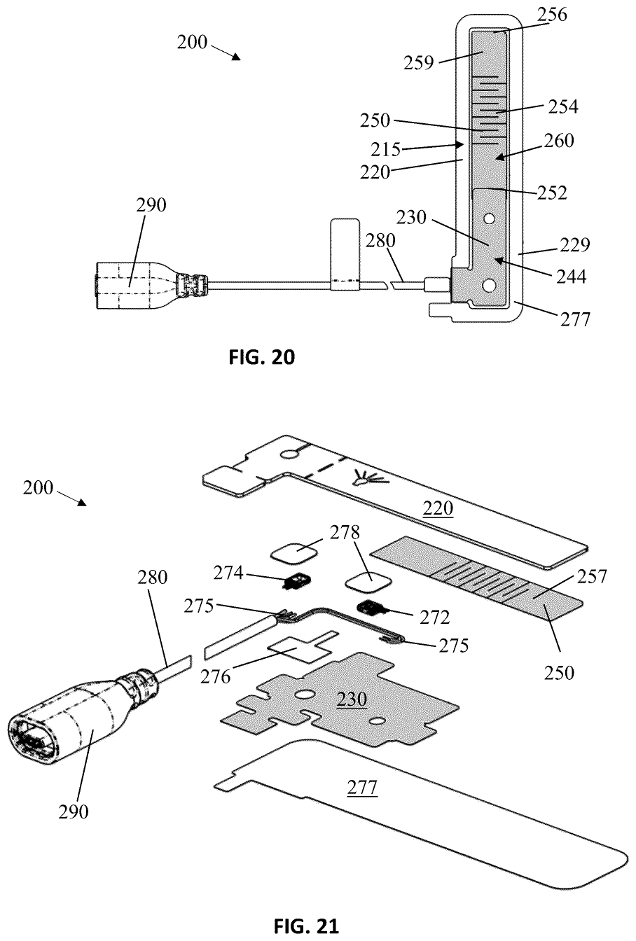

Referring now generally to the figures, a device 100, 200 is configured to be applied to a tissue area 505 of a patient 500 is shown. When applied to tissue area 505, the device at the sensor site can measure SpO₂ of patients of any skin pigmentation, including light skin pigmentation, intermediate skin pigmentation and dark skin pigmentation. The device is preferably a non-invasive pulse oximetry sensor configured for use with a pulse oximeter (not shown). The device generally comprises a sensor assembly 110, 210. In some preferred aspects, the sensor assembly (110, 210) is in electrical communication with a connector assembly (190, 290) via a cable assembly (180, 280) as shown in FIGS. 2A-4, 7-8, 11-12, and 14-21.

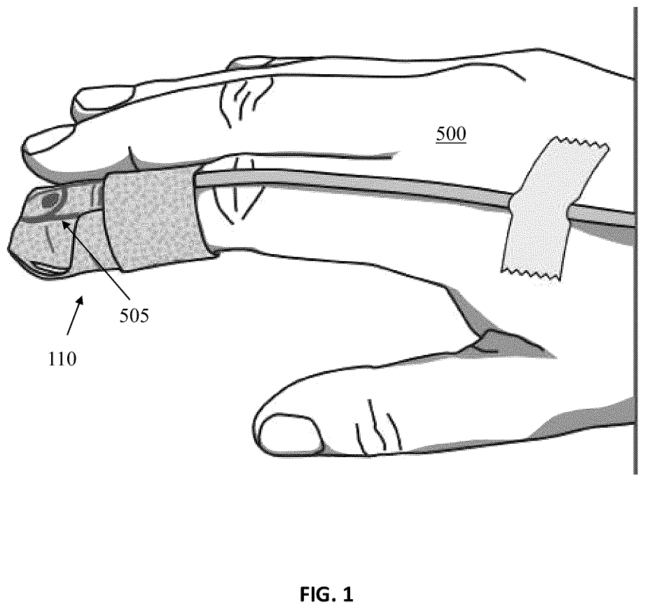

Referring now to FIGS. 1-13, sensor assembly 110 according to certain preferred aspects is illustrated. As shown in FIG. 1, sensor assembly 110 is configured to be applied to tissue area 505 of patient 500. Sensor assembly 110 preferably includes substrate body 120, envelope assembly 130, and transmission assembly 170 having at least one light emitter 172 and at least one light detector 174. In some preferred aspects, sensory assembly 110 is configured to be applied to a digit, such as a fingertip or toe, of patient 500. Sensory assembly 110 can also be applied to other tissue areas of patient 500, such as an earlobe, palm of hand, foot, or other body part that allows proper alignment of at least one light emitter 172 with at least one light detector 174 when applied to patient 500. While the sensor assembly 110 in FIGS. 1-13 is preferably configured for application to an adult or pediatric patient, it is contemplated that sensor assembly 110 can also be applied to an infant or neonate.

Sensor assembly 110 may be a “transmission type” sensor, which is configured to include at least one light emitter 172 and at least one light detector 174 positioned on opposing sides of the sensor site and configured to operably engage with each other. For example, if the sensor site is a fingertip as shown in FIG. 1, sensor assembly is positioned over the patient's fingertip, such that light emitter 172 and light detector 174 are on opposing sides of the patient's fingertip. During operation, light emitter 172 transmits one or more wavelengths of light through the patient's fingertip, and the light received by the light detector 174 is processed to determine various physiological characteristics of the patient, such as SpO₂ and/or pulse rate. In the disclosure herein, it should be understood that the relative locations of the light emitter 172 and light detector 174 may be exchanged. For example, the light detector 174 may be located at the top of the finger proximate the fingernail, and the light emitter 172 may be located underneath the finger. In either arrangement, device 100 will perform in substantially the same manner.

In some other aspects, sensor assembly 110 may be a “reflectance type” sensor, which generally operates under the same general principles as transmission type sensors, except reflectance type sensors include at least one light emitter and at least one light detector that are typically placed on the same side of the sensor site. For example, sensor assembly 110 in a reflectance type sensor configuration may be placed on a patient's forehead, such that the light emitter 172 and the light detector 174 lay in a side-by-side configuration, and the light detector 174 would detect light photons that are reflected back to the light detector 174.

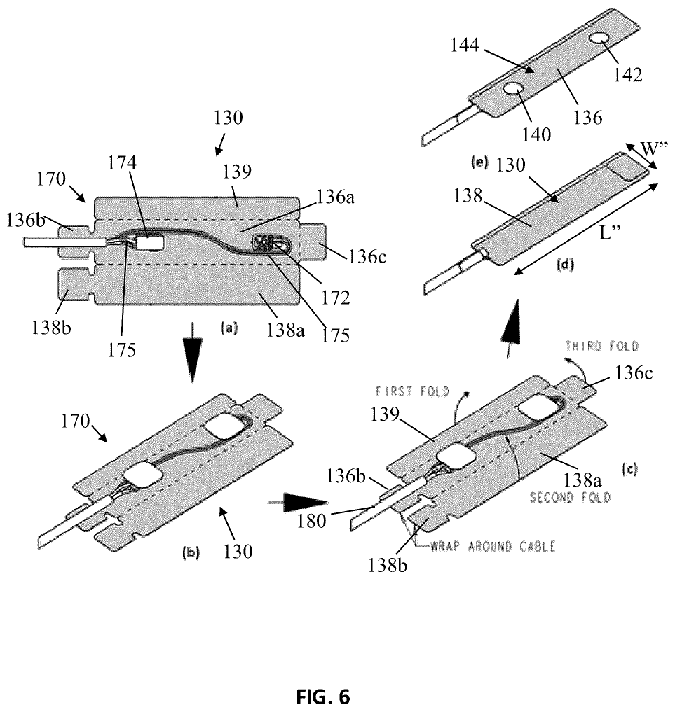

Substrate body 120 includes intermediate portion 124 extending between first end 122 and opposing second end 126. In some aspects, such as shown in FIG. 2A and FIG. 7A, substrate body 120 includes member 121 located proximate first end 122 that is configured to wrap around at least a portion of cable assembly 180. In some other aspects, as shown in FIGS. 2B-2E, it is contemplated that member 121 configured to wrap around at least a portion of cable assembly 180, if present, can be located at different locations on substrate 120. While FIGS. 2A-2E illustrate device 10, various configurations are contemplated in relation to device 100 including envelope assembly 130, one or more light absorption layers 150, a combination of envelope assembly 130 and light absorption layer 150, or any other variation of one or more layers 136, 138 of envelope assembly 130 and/or light absorption layer 150 disclosed herein. As such, when device 100 is operably applied to tissue area 505 of the patient 500, cable assembly 180 may be located at different locations, such as on top of a finger and running towards the top of the hand (for example, FIGS. 2A and 2E), on the bottom of a finger and running towards the bottom of the hand (for example, FIGS. 2B and 2D), and running in a direction perpendicular to the finger (for example, FIG. 2C).

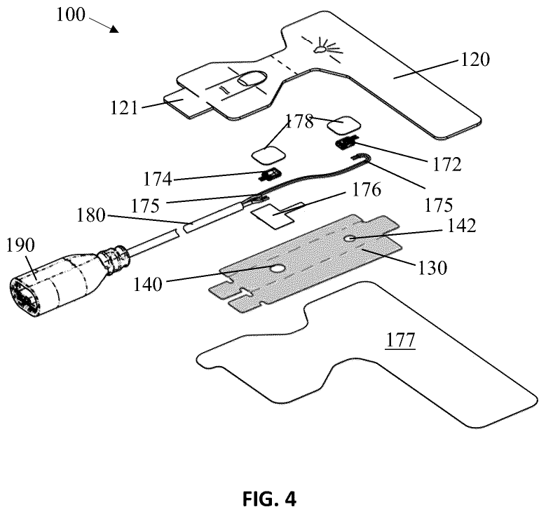

In some preferred aspects, substrate body 120 includes a pair of opposing proximal tabs 123a, 123b located proximate first end 122. Opposing tabs 123a, 123b are preferably configured to extend away from centerline (C), as shown in FIG. 5, which extends between first and second ends 122, 126 of substrate body 120, such that opposing tabs 123a, 123b form an opposing wing configuration with respect to intermediate portion 124. In some preferred aspects, width (W2) of substrate body 120 located proximate opposing tabs 123a, 123b is greater than width (W1) of substrate body 120 located proximate intermediate portion 126. In some preferred aspects, substrate body 120 includes distal tab 125 located proximate second end 126. Distal tab 125 is preferably configured to extend away from centerline (C), such that width (W3) of substrate body 120 located proximate distal tab 125 is greater than width (W1) of substrate body 120 located on the same side of centerline (C) proximate intermediate portion 124. In some preferred aspects, substrate body 120 includes elongated member 128 located proximate second end 126 and located the opposite side of centerline (C), such that elongated member 128 is opposing distal tab 125. In certain aspects, elongated member 128 extends away from substrate body 120, such that width (W4) of substrate body 120 located proximate elongated member 128 is greater than width (W1) of substrate body 120 located on the same side of centerline (C) proximate intermediate portion 124.

In some preferred aspects, substrate body 120 includes opposing tabs 123a, 123b, distal tab 125 and elongated member 128, whereby opposing tabs 123a, 123b, distal tab 125 and elongated member 128 each extend away from centerline (C), such that opposing tabs 123a, 123b form a first opposing wing configuration and distal tab 125 forms a second opposing wing configuration with respect to elongated member 128, in relation to intermediate portion 124. In some preferred aspects, width (W2) of substrate body 120 located proximate opposing tabs 123a, 123b is greater than width (W1) of substrate body 120 located proximate intermediate portion 124, and width (W5) of substrate body 120 located proximate distal tab 125 and elongated tape member 128 is greater than both width (W1) of substrate body 120 located proximate intermediate portion 124 and width (W2) of substrate body 122 located proximate opposing tabs 125a, 125b.

In some preferred aspects, width (W1) of substrate body 120 located proximate intermediate portion 124 is between about 0.35 inches and about 0.70 inches, in some aspects between about 0.40 inches and about 0.65 inches, and in some preferred aspects between about 0.45 inches and about 0.60 inches. In some preferred aspects, intermediate portion 124 proximate width (W1) has a length (L1) between about 0.25 inches and about 1.35 inches, in some aspects between about 0.30 inches and about 0.85 inches, and in some preferred aspects between about 0.35 inches and about 0.65 inches. In some preferred aspects, intermediate portion 124 extends away from centerline (C) in each direction a distance between 0.225 inches and about 0.30 inches, in some aspects between 0.2 inches and about 0.325 inches, and in some preferred aspects between about 0.175 inches and about 0.35 inches.

In some preferred aspects, each of opposing tab 123a, 123b extends away from centerline (C) a distance greater than the distance of intermediate portion 124 extending away from centerline (C) in each direction that is between about 0.125 inches and about 0.425 inches, in some aspects between about 0.15 inches and about 0.415 inches, and in some preferred aspects between about 0.20 inches and about 0.40 inches. In some preferred aspects, width (W2) of substrate body 120 located proximate is opposing tabs 123a, 123b is between about 0.75 inches and about 1.50 inches, in some aspects between about 0.80 inches and about 1.45 inches, and in some preferred aspects between about 0.85 inches and about 1.40 inches. In some preferred aspects, each of opposing tabs 123a, 123b proximate width (W2) has a length (L2) between about 0.35 inches and about 1.4 inches, in some aspects between about 0.40 inches and about 1.3 inches, and in some preferred aspects between about 0.50 inches and about 1.2 inches.

In some preferred aspects, distal tab 125 extends away from centerline (C) a distance greater than the distance of intermediate portion 124 extending away from centerline (C) that is between about 0.2 inches and about 0.575 inches, in some aspects between about 0.25 inches and about 0.55 inches, and in some preferred aspects between about 0.30 inches and about 0.525 inches. In some preferred aspects, width (W3) of substrate body 120 located proximate distal tab 125 has a greater width than width (W1) by at least 0.25 inches and up to about 0.575 inches, in some aspects at least about 0.30 inches and up to about 0.55 inches, and in some preferred aspects at least about 0.35 inches and up to about 0.50 inches. In some preferred aspects, distal tab 125 proximate width (W3) has a length (L3) between about 0.40 inches and about 1.3 inches, in some aspects between about 0.45 inches and about 1.2 inches, and in some preferred aspects between about 0.50 inches and about 1.1 inches.

In some preferred aspects, elongated member 128 extends away from centerline (C) a distance (W4) greater than distance (W1) of intermediate portion 124 extending away from centerline (C) that is between about 2 inches and about 4 inches, in some aspects between about 2.25 inches and about 3.25 inches, and in some preferred aspects between about 2.5 inches and about 3.0 inches. In some preferred aspects, width (W4) of substrate body 120 located proximate elongated member 128 is between about 2.25 inches and about 4.25 inches, in some aspects between about 2.5 inches and about 3.5 inches, and in some preferred aspects between about 2.75 inches and about 3.25 inches. In some preferred aspects, elongated member 128 proximate width (W4) has a length (L4) between about 0.40 inches and about 1.0 inches, in some aspects between about 0.5 inches and about 0.9 inches, and in some preferred aspects between about 0.65 inches and about 0.8 inches.

Substrate body 120 has an axial length (L′) from first end 122 to second end 126 that is preferably between about 1.5 inches and about 3 inches, in some preferred aspects between about 1.75 inches and about 2.75 inches, and in some preferred aspects between about 1.9 inches and about 2.6 inches.

Substrate body 120 preferably comprises a conformable material, such as a bandage type material, cloth material, nonwoven tape material, or the like. In some preferred aspects, substrate body 120 comprises a tear-resistant cloth material, such that substrate body 120 can be placed on the desired tissue area 505 and conformed to the contour of the tissue area 505. In some other preferred aspects, substrate body 120 comprises a tear-resistant, stretchable cloth material, such that substrate body 120 can be placed on the desired tissue area 505 and conformed to the contour of the tissue area 505. In some preferred aspects, substrate body 120 is free of any latex, di-(2-ethylhexyl)phthalate (DEHP) and/or bisphenol A (BPA). In some other preferred aspects, substrate body 120 comprises a medical tape or other wearable tape, such as the nonwoven medical tape commercially available from Solventum™ and 3M™.

Substrate body 120 preferably includes first surface 127 configured to be located away from the tissue area 505 when sensor assembly 110 is applied to patient 500. In some preferred aspects, first surface 127 is substantially free of any adhesive material. As shown in FIGS. 1-2E, 4, 8 and 12, first surface 127 may have one or more indicia 199 related to proper placement of sensor assembly 110 on tissue area 505 of patient 500. For example, first surface 127 may have indicia 199 related to the fold line of sensory assembly 110, digit (e.g., finger or toe) placement, light emitter location, light detector location, or a combination thereof. In some preferred aspects, the one or more indicia 199 provide guidance for proper placement such that there is proper operable engagement of light emitter 172 and light detector 174 when sensor assembly 110 is applied to patient 500.

Substrate body 120 preferably includes second surface 129 configured to be facing towards tissue area 505 when sensor assembly 110 is applied to patient 500. In some preferred aspects, second surface 129 contains an adhesive layer 115. In some preferred aspects, at least a portion of adhesive layer 115 is in direct contact with tissue area 505 when sensor assembly 110 is applied to patient 500. In some preferred aspects, adhesive layer 115 comprises a biocompatible material.

Envelope assembly 130 is preferably disposed relative to second surface 129 of substrate body 120. In some preferred aspects, at least a portion of adhesive layer 115 is located between second surface 129 and envelope assembly 130. Envelope assembly 130 is preferably disposed relative to second surface 129 such that a proximal end 132 is located proximate first end 122 of substrate body 120 and a distal end 134 is located proximate second end 126 of substrate body 120. In some preferred aspects, envelope assembly 130 has a length that is equal to or less than the axial length (L′) of substrate body 120. In some preferred aspects, envelope assembly 130 has a width that is equal to or less than the width (W1) of intermediate portion 124. In some preferred aspects, envelope assembly 130 has an axial length (L″) between about 1.75 inches and about 3.5 inches, in some aspects between about 2.25 inches and about 3.25 inches, and in some other preferred aspects between about 2.3 inches and about 3.0 inches. In some preferred aspects, envelope assembly 130 has a width (W″) between about 0.35 inches and about 0.70 inches, in some aspects between about 0.40 inches and about 0.60 inches, and in some preferred aspects between about 0.45 inches and about 0.55 inches.

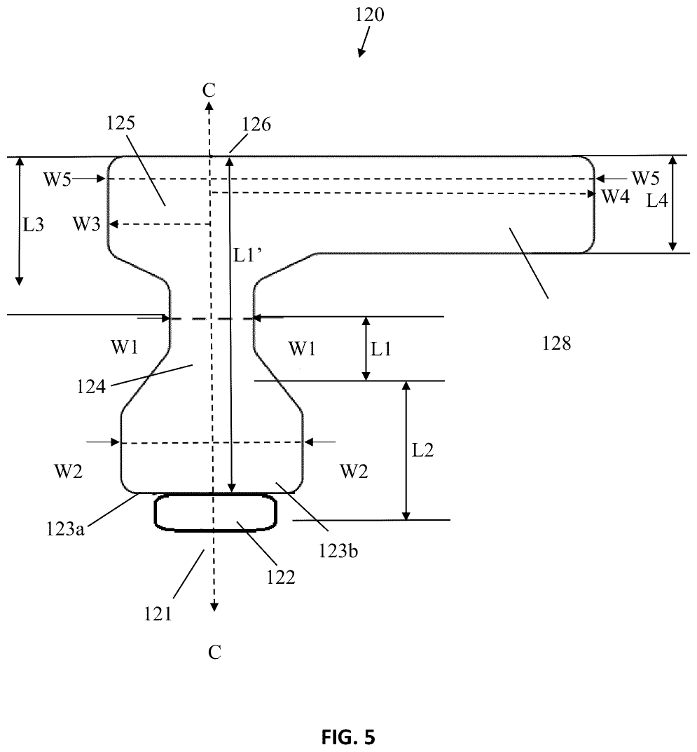

As shown in FIG. 6, envelope assembly 130 preferably comprises a first layer 136 and an opposing second layer 138. Second layer 138 can be configured to be located between second surface 129 of substrate body 120 and first layer 136, such that first layer 136 can be located closer to tissue area 505 than second layer 138. In some preferred aspects, at least a portion of adhesive layer 115 is in direct contact with second layer 138.

First layer 136 preferably has a first aperture 140 spaced apart a distance from a second aperture 142. In some preferred aspects, first aperture 140 is located between first end 122 and intermediate portion 124 of substrate body. In some preferred aspects, second aperture 142 is located between second end 126 and intermediate portion 124 of substrate body. The distance between first and second apertures 140, 142 is preferably between about 0.70 inches and 1.5 inches, in some aspects between 0.75 inches and about 1.4 inches, and in some preferred aspects between about 0.80 inches and about 1.3 inches.

In some preferred aspects, first aperture 140 has a radius that is preferably larger in size than the radius of second aperture 142. In some other aspects, first aperture 140 and second aperture 142 are approximately the same size.

First and second layers 136, 138 can be separate layers. When comprised of separate layers, first and second layers 136, 138 can be configured to be at least partially adhered together to form envelope assembly 130. In some aspects, an adhesive is applied between at least a portion of first and second layers 136, 138.

Envelope assembly 130 is preferably configured from a single sheet of material in a folded configuration to form first and second layers 136, 138. For instance, as shown in FIG. 6, envelope assembly 130 prior to the folded configuration can have two or more sections. In some preferred aspects, envelope assembly 130 prior to the folded configuration has at least three sections, whereby a first section 136a forms at least a portion of first layer 136, a second section 138a folded relative to first section 136a forms at least a portion of second layer 138, and a third section 139 folded relative to first section 136a operably engages with second section 138a to form second layer 138. In some aspects, second section 138a can include a proximal member portion 138b that can operably engage with a portion of cable. In some aspects, first section 136a can include a proximal member portion 136b that can operably engage with a portion of cable. In some aspects, first section 136a can include a distal member portion 136c that can be folded to operably engage with at least a portion of second section 138a and/or third section 139.

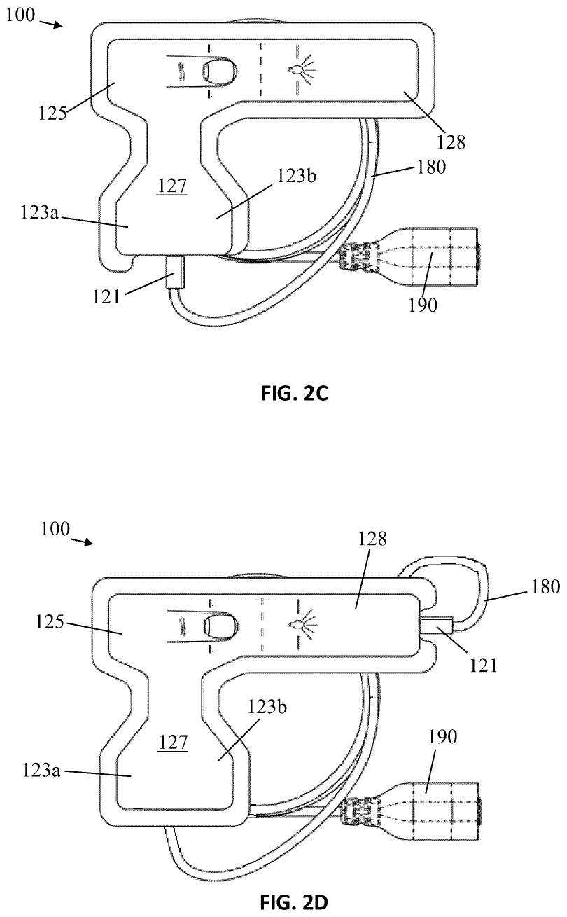

Envelope assembly 130 preferably includes at least one layer that comprises a light absorption material. In some aspects, first layer 136 or second layer 138 comprises a light absorption material. In some preferred aspects, as shown in FIGS. 3-4 and 6, both first layer 136 and second layer 138 of envelope assembly 130 comprise a light absorption material.

Transmission sensor assembly 170 is preferably at least partially contained between first and second layers 136, 138 of envelope assembly 130. Transmission sensor includes at least one light emitter 172 and at least one light detector 174, whereby each of light emitter 172 and light detector 174 are located proximate first and second apertures 140, 142. Light emitter 172 is preferably located proximate first aperture 142, and light detector 174 is preferably located proximate second aperture 140. In some alternative aspects, light emitter 172 is preferably located proximate second aperture 140, and light detector 174 is preferably located proximate first aperture 142. Light emitter 172 is configured to emit at least one wavelength of light into the tissue area 505 of the patient, and light detector 174 is configured to detect at least one wavelength that has been emitted by light emitter 172, transmitted through the tissue area 505 of the patient and received by light detector 174. In some aspects, the at least one wavelength of light emitted by light emitter 172 is a range of wavelengths, and the at least one wavelength received by light detector 174 is the range of wavelengths emitted from light emitter 172. In some preferred aspects, light emitter 172 is configured to emit a first wavelength and a second wavelength into the tissue area 505 of the patient, and light detector 174 is configured to detect the first and second wavelengths that has been emitted by light emitter 172, transmitted through the tissue area 505 of the patient, and received by light detector 174.

In some preferred aspects, the central tendency of first wavelength of light emitted by light emitter 172 is in the range between 620 nm and about 750 nm, more preferably between 630 nm and 690 nm, and even more preferably between 650 nm and 670 nm. In some preferred aspects, the first wavelength of light is at about 660 nm.

In some preferred aspects, the central tendency of second wavelength of light emitted by light emitter 172 is in the range between 780 nm and about 1200, more preferably between 880 nm and about 940 nm, and even more preferably between 900 nm and 920 nm. In some preferred aspects, the second wavelength of light is at about 910 nm.

When sensor assembly 110 is properly applied to tissue area 505 of the patient 500, light detector 174 and light emitter 172 are configured to be positioned on opposing sides of tissue area 505, such that light detector 174 is configured to operably engage with light emitter 172 to detect the at least the first and second wavelengths of light emitted by light emitter 172 and transmitted through the sensor site of patient 500, and light detector 174 can be configured to generate at least one electrical signal relating to the at least one physiological parameter of patient 500. In some preferred aspects, the at least one signal relates to an SpO₂ measurement of the patient. In some preferred aspects, the at least one signal relates to an SpO₂ measurement and pulse rate of the patient. In some preferred aspects, when sensor assembly 110 is properly applied to tissue area 505 of the patient 500, light detector 174 is configured to be substantially aligned with light emitter 172 for the detection of the at least first and second wavelengths of light emitted by light emitter 172 and transmitted through the sensor site the patient. The alignment between light emitter 172 and light detector 174 may include some degree of offset without substantially affecting the detection of the at least the first and second wavelengths of light emitted by light emitter 172 and transmitted through the sensor site of patient 500.

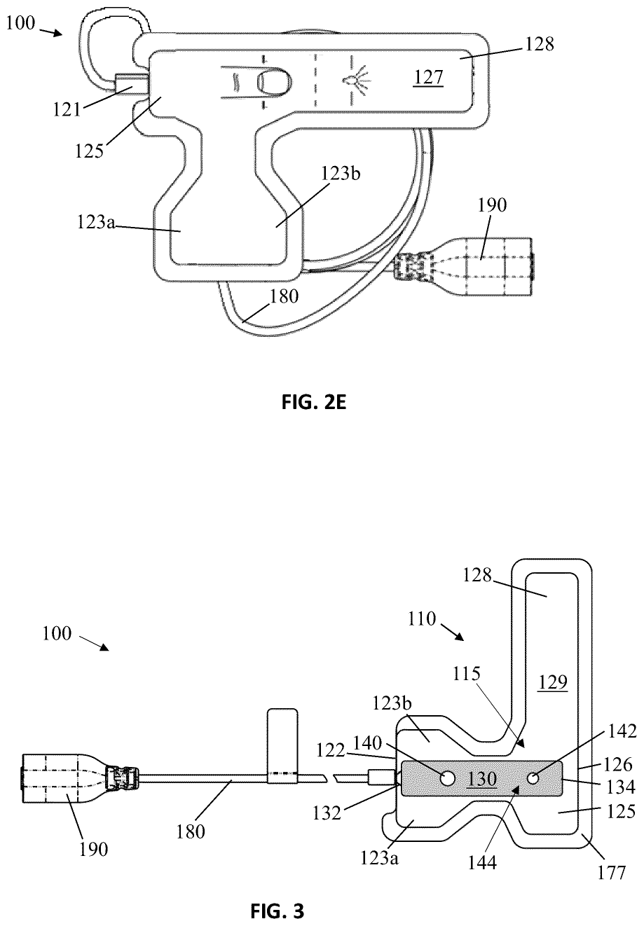

Referring now to FIGS. 3-4, sensor assembly 110 according to certain preferred aspects is shown, whereby envelope assembly 130 is provided between substrate body 120 and tissue area 505 of the patient 500. Envelope assembly 130 is preferably disposed on the second surface 129 of substrate body 120 located proximate intermediate portion 124 in a configuration such that proximal and distal ends 132, 134 of envelope assembly 130 extend between first and second ends 122, 126 of substrate body 120 with light emitter 172 and light detector 174 being disposed on opposite sides of intermediate portion 124 of substrate body 120. In some preferred aspects, light emitter 172 is located between intermediate portion 124 and second end 126 and light detector 174 is located between intermediate portion 124 and first end 122. In some alternative aspects, light detector 174 is located between intermediate portion 124 and second end 126 and light emitter 172 is located between intermediate portion 124 and first end 122. In either configuration, sensor assembly 110 is configured to be folded at a center position of intermediate portion 124 when applied to tissue area 505 of the patient 500, such that light emitter 172 and light detector 174 being provided on opposite sides of intermediate portion 124 enables proper operable engagement during normal operational use.

In some other aspects, such as shown in FIG. 7B, envelope assembly 130 provided in a configuration such that proximal and distal ends 132, 134 of envelope assembly are configured to extend between distal tab 125 and at least a portion of elongated member 128. In this configuration, light emitter 172 and light detector 174 can be disposed on opposite sides of envelope assembly 130. In some aspects, light emitter 172 is located proximate the area of distal tab 125 and light detector is located the area of a portion of elongated member 128. In some alternative aspects, light detector 174 is located the area of a distal tab 125 and light emitter 172 is located an area of elongated member 128. In either configuration, sensor assembly 110 is configured to be folded at a center position between distal end 132 and elongated member 128 when applied to tissue area 505 of the patient 500, such that light emitter 172 and light detector 174 being provided on opposite sides within envelope assembly 130 enables the operable engagement of light emitter 172 and light detector 174 during normal operational use. In some aspects, light detector 174 and light emitter can be configured to be substantially aligned for the detection of the at least first and second wavelengths of light emitted by light emitter 172 and transmitted through the sensor site the patient, although alignment between light emitter 172 and light detector 174 may include some degree of offset without substantially affecting the detection of the at least the first and second wavelengths of light emitted by light emitter 172 and transmitted through the sensor site of patient 500.

In some preferred aspects, at least a portion of adhesive layer 115 can be provided between substrate body 120 and second layer 138 of envelope assembly 130. In some preferred aspects, adhesive layer 115 can be provided on second surface 129 of substrate body 120, such that at least a portion of adhesive layer 115 is provided between substrate body 120 and second layer 138 of envelope assembly 130, and at least a portion of adhesive layer 115 that does not operably engage with envelope assembly 130 can be operably applied to tissue area 505 of patient 500. In some preferred aspects, first layer 136 of envelope assembly 130 includes adhesive layer 144 for applying sensor assembly 110 to tissue area 505 of patient 500. In some preferred aspects, sensor assembly 110 includes adhesive layer 144 on first layer 136 of envelope assembly 130 and also at least a portion of adhesive layer 115 that is not operably engaged with envelope assembly 130, such that at least a portion of second surface 129 of substrate body 130 and first layer 136 of envelope assembly each include an adhesive for applying sensor assembly 110 to the tissue area 505 of the patient 500 to maintain proper placement of sensor assembly 110 on the patient and operable engagement of light emitter 172 and light detector 174 during non-motion and motion activities.

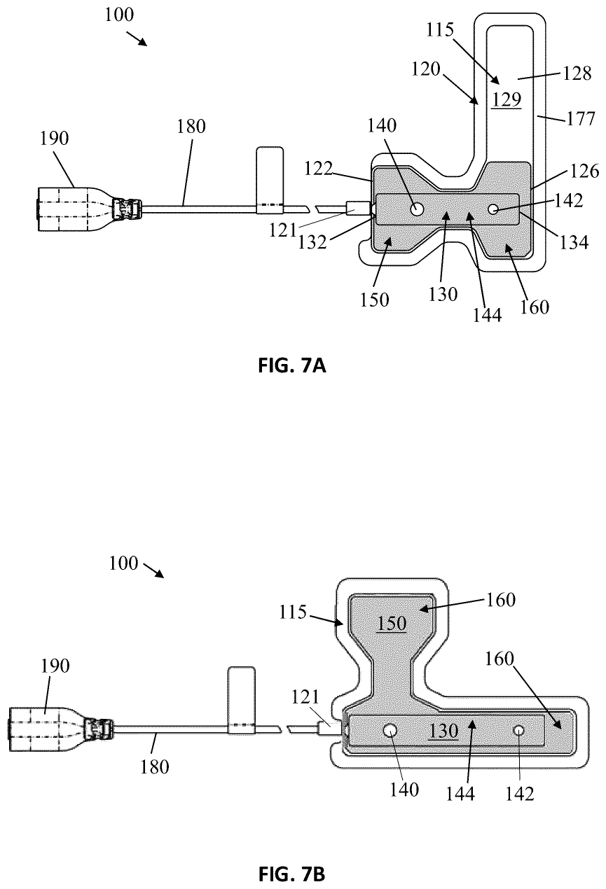

Referring now to FIGS. 7A-13, in some exemplary aspects, sensor assembly 110 preferably includes at least one light absorption layer 150. Light absorption layer 150 preferably comprises a light absorption material. In some preferred aspects, such as shown in FIGS. 7A and 8A, light absorption layer 150 is preferably configured to have a shape that is substantially similar to at least a portion of the shaped configuration of substrate body 120. In some preferred aspects, such as shown in FIGS. 7B and 8B, light absorption layer 150 is preferably configured to have a shape that is substantially similar to the shaped configuration of substrate body 120. In some exemplary aspects, light absorption layer 150 can be configured to have a substantially rectangular shape, square shape, oval shape, bow-tie shape, hour-glass shape, butterfly shape, oblong, or other geometrical shape that spans at least a portion of the area between first and second ends 122, 126 of substrate body 120, such as shown in FIGS. 7A and 8A. In some other aspects, the light absorption layer 150 can be configured to have a geometric shape that spans not only a portion of the area between first and second ends 122, 126 of substrate body 120, but also at least a portion of the area between distal tab 125 and elongated member 128 of substrate body 120, such as shown in FIGS. 7B and 8B.

In some exemplary aspects, at least a portion of the shaped configuration of light absorption layer 150 is substantially similar to at least the pair of opposing proximal tabs 123a, 123b of substrate body 120. In some other exemplary aspects, at least a portion of the shaped configuration of light absorption layer 150 is substantially similar to at least intermediate portion 124 of substrate body 120. In some exemplary aspects, at least a portion of the shaped configuration of light absorption layer 150 is substantially similar to at least distal tab 125 of substrate body 120. In some exemplary aspects, at least a portion of the shaped configuration of light absorption layer 150 is substantially similar to at least a portion of elongated tab 128 of substrate body 120, or alternatively the entirety of elongated tab 128 of substrate body 120. In some preferred exemplary aspects, at least a portion of the shaped configuration of light absorption layer 150 is substantially similar to at least the pair of opposing proximal tabs 123a, 123b, intermediate portion 124, distal tab 125, at least a portion of elongated member 128, or a combination thereof. In some preferred aspects, the portion of elongated member 128 that light absorption layer 150 is substantially similar to reflects the substantial similarity to distal tab 125. In some preferred exemplary aspects, at least a portion of the shaped configuration of light absorption layer 150 is substantially similar to each of the pair of opposing proximal tabs 123a, 123b, intermediate portion 124, distal tab 125 and at least a portion of elongated member 128.

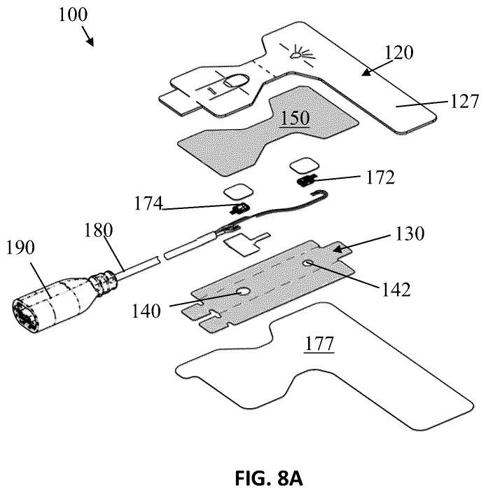

In some preferred aspects, as shown in FIGS. 9A-9B, light absorption layer 150 includes intermediate portion 154 extending between first end 152 and opposing second end 156. In some aspects, light absorption layer 150 includes a pair of opposing proximal tabs 153 located proximate first end 152. Opposing proximal tabs 153 are preferably configured to extend away from centerline (C′), which extends between first and second ends 152, 156 of light absorption layer 150, such that opposing tabs 153 form an opposing wing configuration with respect to intermediate portion 154. In some preferred aspects, width (W2′) of light absorption layer 150 located proximate opposing proximate tabs 153 is greater than width (W1′) located proximate intermediate portion 154. In some preferred aspects, light absorption layer 150 includes opposing distal tabs 155 located proximate second end 156. Distal tabs 155 are preferably configured to extend away from centerline (C′), such that opposing distal tabs 155 form an opposing wing configuration with respect to intermediate portion 154. In some preferred aspects, width (W3′) of light absorption layer 150 located proximate opposing distal tabs 155 is greater than width (W1′) located proximate intermediate portion 124. In some preferred aspects, width (W3′) of light absorption layer 150 located proximate opposing distal tabs 155 is greater than width (W2′) of light absorption layer 150 located proximate opposing proximate tabs 153.

In some preferred aspects, light absorption layer 150 includes opposing proximal tabs 153 and distal tabs 155 relative to intermediate portion 154, whereby each of opposing proximal tabs 153 and each of opposing distal tabs 155 extend away from centerline (C′), such that opposing proximal tabs 153 form a first opposing wing configuration and opposing distal tabs 155 form a second opposing wing configuration in relation to intermediate portion 154.

In some exemplary aspects, light absorption layer 150 has a surface area that substantially overlaps second surface 129 of substrate body 130 including at least a portion of elongated member 128, in some aspects substantially the entire portion of elongated member 128, and in some alternative aspects only the portion of elongated member 128 that does not extend beyond the width (W3) of distal tab 125 relative to elongated member 128. In some other exemplary aspects, second surface 129 has a surface area that is slightly larger than the surface area of light absorption layer 150, excluding the surface area relative to elongated member 128 that extends beyond the width (W3) of distal tab 125 relative to elongated member 128 portion of substrate body 130. In some other exemplary aspects, the surface area of light absorption layer 150 relative to the surface area of second surface 129 is slightly smaller, such that there is a gap between the perimeter of light absorption layer 150 and perimeter of the second surface 129 that, in some aspects, is less than 2 mm, in some aspects less than 1.5 mm, and in some other aspects less than 1 mm, notwithstanding at least a portion of elongated member 128 portion of second surface 129.

In some preferred aspects, width (W1′) of light absorption layer 150 located proximate intermediate portion 154 is between about 0.35 inches and about 0.70 inches, in some aspects between about 0.40 inches and about 0.65 inches, and in some preferred aspects between about 0.45 inches and about 0.60 inches. In some preferred aspects, intermediate portion 154 has a length (L4″) between about 0.25 inches and about 1.35 inches, in some aspects between about 0.30 inches and about 0.85 inches, and in some preferred aspects between about 0.35 inches and about 0.65 inches. In some preferred aspects, intermediate portion 154 extends away from centerline (C′) in each direction a distance between 0.225 inches and about 0.30 inches, in some aspects between 0.2 inches and about 0.325 inches, and in some preferred aspects between about 0.175 inches and about 0.35 inches.

In some preferred aspects, each of opposing proximal tabs 153 of light absorption layer 150 extends away from centerline (C′) a distance greater than the distance of intermediate portion 154 extending away from centerline (C′) in each direction that is between about 0.125 inches and about 0.425 inches, in some aspects between about 0.15 inches and about 0.415 inches, and in some preferred aspects between about 0.20 inches and about 0.40 inches. In some preferred aspects, width (W2′) of light absorption layer 150 located proximate opposing proximal tabs 153 is between about 0.75 inches and about 1.50 inches, in some aspects between about 0.80 inches and about 1.45 inches, and in some preferred aspects between about 0.85 inches and about 1.40 inches. In some preferred aspects, each of opposing proximal tabs 153 located proximate width (W2′) has a length (L2″) between about 0.35 inches and about 1.4 inches, in some aspects between about 0.40 inches and about 1.3 inches, and in some preferred aspects between about 0.50 inches and about 1.2 inches.

In some preferred aspects, opposing distal tabs 155 extend away from centerline (C′) a distance greater than the distance of intermediate portion 154 extending away from centerline (C′) that is between about 0.2 inches and about 0.575 inches, in some aspects between about 0.25 inches and about 0.55 inches, and in some preferred aspects between about 0.30 inches and about 0.525 inches. In some preferred aspects, width (W3′) of light absorption layer 150 located proximate distal tabs 155 is greater width than width (W1′). In some preferred aspects, width (W3′) of light absorption layer 150 located proximate opposing distal tabs 155 is between about 0.90 inches and about 1.70 inches, in some aspects between about 1.1 inches and about 1.60 inches, and in some preferred aspects between about 1.2 inches and about 1.55 inches. In some preferred aspects, each of opposing distal tabs 155 located proximate width (W3′) has a length (L3″) between about 0.40 inches and about 1.3 inches, in some aspects between about 0.45 inches and about 1.2 inches, and in some preferred aspects between about 0.50 inches and about 1.1 inches.

Light absorption layer 150 has an axial length (L1″) from first end 152 to second end 156 that is preferably between about 1.5 inches and about 3 inches, in some preferred aspects between about 1.75 inches and about 2.75 inches, and in some preferred aspects between about 1.9 inches and about 2.6 inches.

While the foregoing disclosure related to the exemplary shaped configuration in the figures, it is contemplated that by this disclosure that light absorption layer 150 can be configured to have a shape that surrounds at least aperture 140 and spans at least a portion of the area between apertures 140, 142. In some other preferred aspects, light absorption layer 150 can be configured to have a shape that surrounds at least aperture 142 and spans at least a portion of the area between apertures 140, 142. In some other preferred aspects, light absorption layer 150 can be configured to have a shape that surrounds each of apertures 140, 142 and spans at least a portion of the area between apertures 140, 142. In each of the foregoing aspects, light absorption layer can have various geometrical shapes, including at least a substantially rectangular shape, square shape, oval shape, bow-tie shape, hour-glass shape, butterfly shape, oblong, or other geometrical shape.

Light absorption layer 150 preferably can have an adhesive layer 160 provided on at least one side. In some aspects, light absorption layer 150 can have an adhesive layer 160 provided on both sides.

Referring now to FIGS. 7-8, in some exemplary aspects, light absorption layer 150 can be provided between substrate body 120 and envelope assembly 130 with envelope assembly 130 provided between substrate body 120 and tissue area 505 of the patient 500. In this configuration, light absorption layer 150 is provided between substrate body 120 and second layer 138 of envelope assembly 130, such that light absorption layer 150 and second layer 138 are provided between substrate body 120 and transmission assembly 170 that includes light emitter 172 and light detector 174. In some preferred aspects, adhesive layer 115 can be provided between substrate body 120 and first surface 157 of light absorption layer 150. In some preferred aspects, second surface 159 of light absorption layer 150 includes adhesive layer 160.

Light absorption layer 150 is preferably disposed on second surface 129 of substrate body 120, such that intermediate portion 154 of light absorption layer 150 is aligned and substantially overlaps with intermediate portion 124 of substrate body 120, opposing proximal tabs 153 of light absorption layer 150 are aligned and substantially overlap with opposing proximal tabs 123 of substrate body 120, and opposing distal tabs 155 of light absorption layer 150 are aligned and substantially overlap with distal tab 125 and at least a portion of elongated member 128 of substrate body 120.

Envelope assembly 130 is preferably disposed on second surface 159 of light absorption layer 150 located proximate intermediate portion 154 in a configuration such that proximal and distal ends 132, 134 of envelope assembly 130 extend between first and second ends 152, 156 of light absorption layer 150 with light emitter 172 and light detector 174 being disposed on opposite sides of intermediate portion 154 of light absorption layer 150. In some preferred aspects, light emitter 172 is located between intermediate portion 154 and second end 156 and light detector 174 is located between intermediate portion 154 and first end 152. In some alternative aspects, light detector 174 is located between intermediate portion 154 and second end 156 and light emitter 172 is located between intermediate portion 154 and first end 152. In either configuration, sensor assembly 110 is configured to be folded at a center position of intermediate portion 124 of substrate body 120 and intermediate portion 154 of light absorption layer 150, when applied to tissue area 505 of the patient 500, such that light emitter 172 and light detector 174 being provided on opposite sides of intermediate portion 124 of substrate body 120 and intermediate portion 154, which enables the operable engagement of light emitter 172 and light detector 174 during normal operational use.

The patient engagement portions of light absorption layer 150 and envelope assembly 130 preferably include adhesive for applying sensor assembly 110 to the tissue area 505 of the patient 500 to maintain proper placement of sensor assembly 110 on the patient and proper operable engagement of light emitter 172 and light detector 174 during non-motion and motion activities.

In some preferred aspects, at least a portion of adhesive layer 160 can be provided between light absorption layer 150 and second layer 138 of envelope assembly 130. In some preferred aspects, adhesive layer 160 can be provided on second surface 159 of light absorption layer 150, such that at least a portion of adhesive layer 160 is provided between light absorption layer 150 and second layer 138 of envelope assembly 130, and at least a portion of adhesive layer 160 that does not operably engage with envelope assembly 130 can be operably applied to tissue area 505 of patient 500. In some preferred aspects, first layer 136 of envelope assembly 130 includes adhesive layer 144 for applying sensor assembly 110 to tissue area 505 of patient 500. In some preferred aspects, sensor assembly 110 includes adhesive layer 144 on first layer 136 of envelope assembly 130 and also at least a portion of adhesive layer 160 that is not operably engaged with envelope assembly 130, such that at least a portion of second surface 159 of substrate body 130 and first layer 136 of envelope assembly each include an adhesive for applying sensor assembly 110 to the tissue area 505 of the patient 500 to maintain proper placement of sensor assembly 110 on the patient for operable engagement of light emitter 172 and light detector 174 during non-motion and motion activities. In some aspects, at least a portion of adhesive layer 115 that is not operably engaged with envelope assembly 130 or light absorption layer 150 can operably engage tissue area 505 of patient 500 during normal operational use.

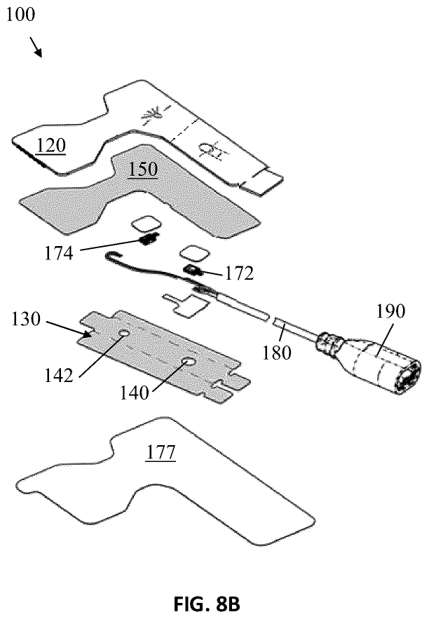

In some aspects, envelope assembly 130 is constructed from a separate sheet of material than light absorption layer 150, such as shown in FIG. 8. In some other preferred aspects, as shown in FIG. 10, envelope assembly 130 and light absorption layer 150 are constructed from a common sheet of material. In exemplary aspects, junction 158 operably connects distal end 134 of envelope assembly 130 and first end 152 of light absorption layer 150. In some preferred aspects, junction 158 operably connects first section 136a proximate distal end 134 of envelope assembly 130 and first end 152 of light absorption layer 150, such that light absorption layer 150 replaces distal member portion 136c of first section 136a. Light absorption layer 150 and envelope assembly 130 can be folded relative to each other along junction 158, such that light absorption layer 150 can be provided between substrate body 120 and envelope assembly 130, as shown in FIG. 7.

Light absorption layer 150 preferably can have an adhesive layer 160 provided on at least one side. In some aspects, light absorption layer 150 can have an adhesive layer 160 provided on both sides, such that adhesive layer 160 provided on first surface 157 of light absorption layer 150 operably engages adhesive layer 115 of substrate body 120.

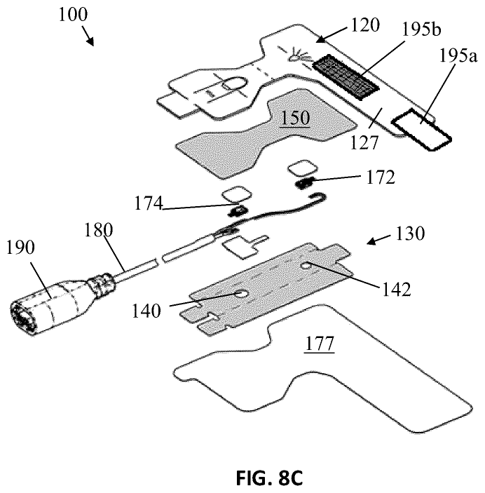

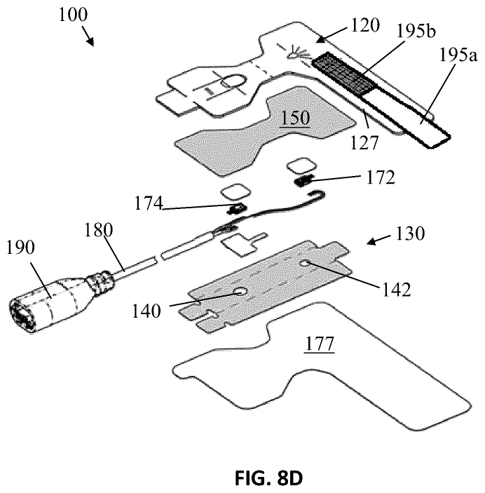

Referring now to FIGS. 11-12A, in some exemplary aspects, envelope assembly 130 can be provided between substrate body 120 and light absorption layer 150 with light absorption layer 150 provided between substrate body 120 and tissue area 505 of the patient 500. In this configuration, envelope assembly 130 is provided between substrate body 120 and first surface 157 of light absorption layer 150, such that transmission assembly 170 that includes light emitter 172 and light detector 174 is provided between substrate body 120 and light absorption layer 150. In some preferred aspects, adhesive layer 115 can be provided between substrate body 120 and second layer 138 of envelope assembly 150 and at least a portion of first surface 157 of light absorption layer 150. Adhesive layer 144 is preferably provided between first layer 136 of envelope assembly 130 and at least a portion first surface 175 of light absorption layer 150. In some aspects, second surface 159 of light absorption layer 150 includes adhesive layer 160.

Envelope assembly 130 is preferably disposed on the second surface 129 of substrate body 120 located proximate intermediate portion 124 in a configuration such that proximal and distal ends 132, 134 of envelope assembly 130 extend at least a portion between first and second ends 122, 126 of substrate body 120 with light emitter 172 and light detector 174 being disposed on opposite sides of intermediate portion 124 of substrate body 120. In some preferred aspects, light emitter 172 is located between intermediate portion 124 and second end 126 and light detector 174 is located between intermediate portion 124 and first end 122. In some alternative aspects, light detector 174 is located between intermediate portion 124 and second end 126 and light emitter 172 is located between intermediate portion 124 and first end 122. In either configuration, sensor assembly 110 is configured to be folded at a center position of intermediate portion 124 when applied to tissue area 505 of the patient 500, such that light emitter 172 and light detector 174 being provided on opposite sides of intermediate portion 124, which enables the operable engagement of light emitter 172 and light detector 174 during normal operational use.

Light absorption layer 150 is preferably disposed on second surface 129 of substrate body 120, such that in some preferred aspects intermediate portion 154 of light absorption layer 150 is aligned and substantially overlaps with first layer 136 of envelope assembly 130, opposing proximal tabs 153 of light absorption layer 150 are aligned and substantially overlap with opposing proximal tabs 123 of substrate body 120, and opposing distal tabs 155 of light absorption layer 150 are aligned and substantially overlap with distal tab 125 and at least a portion of elongated member 128 of substrate body 120.

Light absorption layer 150 preferably includes first and second apertures 151. In some preferred aspects, first aperture 151a is configured to operably align with first aperture 140 of envelope assembly, and second aperture 151b is configured to operably align with second aperture 142 of envelope assembly. The alignment of apertures 151 of light absorption layer 150 with first and second apertures 140, 142 of envelope assembly allows one or more wavelengths of light to be emitted by light emitter 172 and received by light detector 174 during normal operational use.

In some preferred aspects, at least a portion of adhesive layer 115 can be provided between substrate body 120 and second layer 138 of envelope assembly 130. In some preferred aspects, adhesive layer 115 can be provided on second surface 129 of substrate body 120, such that at least a portion of adhesive layer 115 is provided between substrate body 120 and second layer 138 of envelope assembly 130, and at least a portion of adhesive layer 115 that does not operably engage with envelope assembly 130 is provided between substrate body 120 and a portion of first surface 157 of light absorption layer 150. In some preferred aspects, first layer 136 of envelope assembly 130 includes adhesive layer 144 that operably engages at least a portion of first surface 157 of light absorption layer 150. In some preferred aspects, sensor assembly 110 includes adhesive layer 144 on first layer 136 of envelope assembly 130 and also at least a portion of adhesive layer 115 that is not operably engaged with envelope assembly 130, such that at least a portion of second surface 129 of substrate body 130 and first layer 136 of envelope assembly each include an adhesive for operably engaging first surface 157 of light absorption layer 150.

Light absorption layer 150 preferably can have adhesive layer 160 provided on second surface 159 for applying sensor assembly 110 to the tissue area 505 of the patient 500 to maintain proper placement of sensor assembly 110 on the patient and operable engagement of light emitter 172 and light detector 174 during non-motion and motion activities. In some aspects, light absorption layer 150 can have an adhesive layer 160 also provided on first surface 157, such that adhesive layer 160 provided on first surface 157 of light absorption layer 150 operably engages adhesive layer 115 of substrate body 120 and/or adhesive layer 144 of envelope assembly 130.

In some alternative exemplary aspects, such as shown in FIG. 12B, first light absorption layer 150a can be provided between substrate body 120 and second light absorption layer 150b, such that transmission assembly 170 is located between first and second light absorption layers 150a, 150b, and first and second light absorption layers 150a, 150b being provided between substrate body 120 and tissue area 505 of the patient 500 when applied to patient 500. In this configuration, envelope assembly 130 is comprised of light absorption layers 150a, 150b, such that adhesive layer 115 can be provided between substrate body 120 and first light absorption layer 150a. In this alternative configuration, adhesive layer 144 can be provided between at least a portion of first and second light absorption layers 150a, 150b. The opposite surface of second light absorption layer 150b can include adhesive layer 160, which would operably engage with tissue area 505 of patient 500. Similar to device 100 illustrated in FIG. 12A, light emitter 172 and light detector 174 can be located between intermediate portion 124 and either of first and second ends 122, 124. In either configuration, sensor assembly 110 is configured to be folded at a center position of intermediate portion 124 when applied to tissue area 505 of the patient 500, such that light emitter 172 and light detector 174 being provided on opposite sides of intermediate portion 124 enables proper operable engagement during normal operational use.

In the configuration shown in FIG. 12B, second light absorption layer 150b preferably includes first and second apertures 151 that operably engage with light emitter 172 and light detector 174 allowing one or more wavelengths of light to be emitted by light emitter 172 and received by light detector 174 during normal operational use. In some preferred aspects, first and second apertures 151 are substantially aligned with light emitter 172 and light detector 174 to allow the one or more wavelengths of light being emitted by light emitter 172 to be received by light detector 174 during normal operational use.

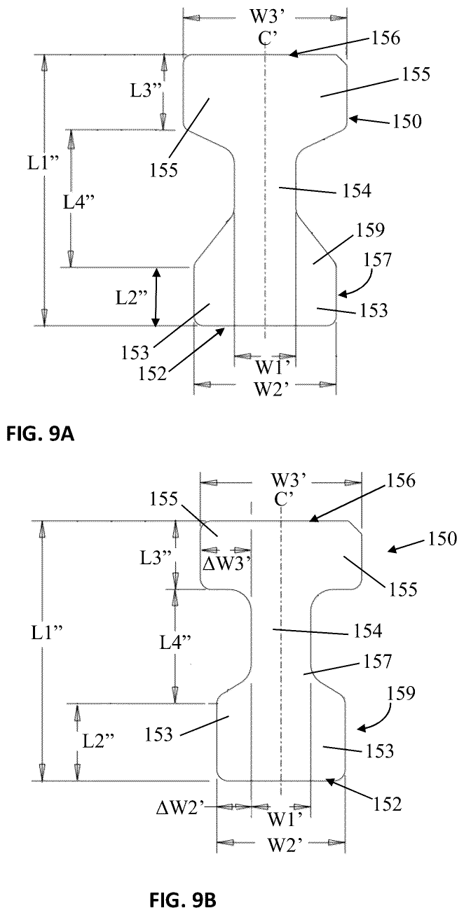

In some exemplary aspects, envelope assembly 130 is constructed from a separate sheet of material than light absorption layer 150, such as shown in FIG. 12. In some other preferred aspects, as shown in FIG. 13, envelope assembly 130 and light absorption layer 150 are constructed from a common sheet of material. In exemplary aspects, junction 158 operably connects distal end 134 of envelope assembly 130 and first end 152 of light absorption layer 150. In some preferred aspects, junction 158 operably connects first section 136a proximate distal end 134 of envelope assembly 130 and first end 152 of light absorption layer 150, such that light absorption layer 150 replaces distal tab portion 136c of first section 136a. Light absorption layer 150 and envelope assembly 130 can be folded relative to each other along junction 158, such that envelope assembly 130 can be provided between substrate body 120 and light absorption layer 150 envelope assembly 130, as shown in FIG. 11. This exemplary configuration maintains the operable engagement of apertures 151 of light absorption layer 150 with first and second apertures 140, 142 of envelope, which allows one or more wavelengths of light to be emitted by light emitter 172 and received by light detector 174 during normal operational use.

In some exemplary aspects, envelope assembly 130 can be comprised of two light absorption layers 150 that contains transmission assembly 170 between the two light absorption layers 150. For instance, a first light absorption layer 150 can be provided between substrate body 120 and light emitter 172 and light detector 174, and a second light absorption layer 150 can be provided on the opposite side of light emitter 172 and light detector 174 as the first light absorption layer 150, such that the second light absorption layer 150 is configured for operable engagement with the tissue area 505 of the patient 500. In some preferred aspects, an adhesive layer can be provided between the first and second light absorption layers 150.

In yet some other exemplary aspects, substrate body 120 can include a light absorption material. In some aspects, a light absorption layer 150 can be provided on first surface 127 of substrate body 120.

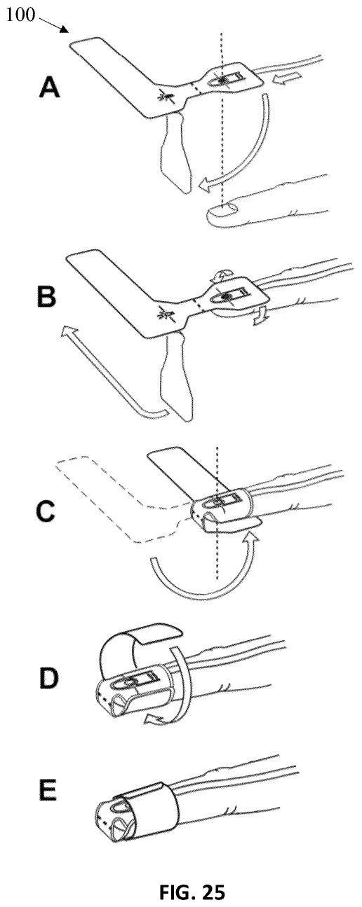

In some exemplary aspects, substrate body 120 and/or light absorption layer 150 can include proximal tabs and at least one distal tab, which are configured for operable engagement with tissue area 505 of patient during normal use. Referring now to FIG. 25, in some exemplary aspects, sensor assembly 110 is applied to tissue area 505 of patient 500 utilizing proximal tabs 123 and at least one distal tab 125. The application of sensor assembly 110 preferably includes applying elongated member 128 to tissue area 505 of patient 500. Sensor assembly 110 may contain release liner 177 that operably engages patient engagement adhesive of sensor assembly 110. Release liner 177 can be partially or completely peeled away from sensor assembly 110, as shown in step (A) of FIG. 25. The tissue area 505 of patient 500 (shown as a fingertip in steps (A)-(E)) can be placed with respect to the tissue engagement surface of sensor assembly 110. In some preferred aspects, the patient's fingernail is placed against the tissue engagement surface located proximate the first end 122 of sensor assembly 110, such that the fingertip is properly operably engaged with the aperture pertaining to the light emitter or light detector. Opposing proximal tabs 123 can be wrapped around the side of the finger, such as illustrated in step (B). If the release liner 177 has not been completely removed, the release liner 177 can be completely removed. Second end 126 of sensor assembly 110 can be folded to the other side of the finger, as shown in step (C), such as to the bottom portion of the finger if the first end was applied to the top portion of the finger or vice versa. The light emitter 172 and light detector 174 can be operably engaged, such as by use of one or more indicia. Distal tab 125 can be wrapped around the side of the finger towards the corresponding proximal tab 123, as shown in step (D). In some preferred aspects, distal tab 125 overlaps with at least a portion of the corresponding proximal tab 123. Elongated member 128 can be wrapped around tissue area 505 as shown in steps (D) and (E), such as the finger. In some preferred aspects, elongated member 128 wrapped around tissue area 505, such as a finger or toe, overlaps each of opposing proximal tabs 123 and distal tab 125. In some preferred aspects, elongated member 128 wrapped around tissue area 505, such as a finger or toe, overlaps another portion of elongated member 128, such as shown in step (E).

In some aspects, tape, such as medical tape, can be applied to a portion of cable assembly 180 independently from sensor assembly 110 to ensure the integrity of proper operable engagement of light emitter 172 and light detector 174 is maintained during normal operational use. Tape may also be used to keep substrate 120 in proper positioning, such as applying tape to maintain substrate 120 in direct contact with at least a portion of the tissue area 505 of the patient 500.

In some other aspects, a retention mechanism 195 can be utilized to maintain the patient monitoring sensor in direct contact with the tissue area of the patient. Retention mechanism 195 can be in direct contact with substrate body 120. In some preferred aspects, retention mechanism 195 is located on first surface 127, such that retention mechanism 195 is configured to be located away from the tissue area 505 when sensor assembly 110 is applied to patient 500. An exemplary retention mechanism 195 is illustrated in FIG. 8C, wherein retention mechanism 195 includes hook-and-loop (e.g., Velcro™), wherein a first portion of retention mechanism 195a is operably attached to the end of elongated member 128 and a second portion of retention mechanism 195b that is capable of operably engaging with the first portion of retention mechanism 195a is operably attached to first surface 127, such as in the area of tab 125, another portion of elongated member 128, or a combination thereof. While FIG. 8C shows each portion 195a, 195b of retention mechanism 195 as separate distinct pieces of material, it is contemplated that retention mechanism 195 may comprise one or more portions in relation to one or more pieces of material, such as the exemplary retention mechanism 195 shown in FIG. 8D. In some aspects, retention mechanism 195 can be an integral part of substrate body 120. In some aspects, retention mechanism 195 is an integral part of first surface 127, second surface 129, or a combination thereof. In some other aspects, retention mechanism 195 is a separate component from substrate body 120. It is contemplated that retention mechanism 195 can include various configurations, including, but not limited to, tape, medical tape, adhesive tape, magnets, self-adhering elastic bandage, posey wrap, elastic material, flexible bistable spring band, and the like.

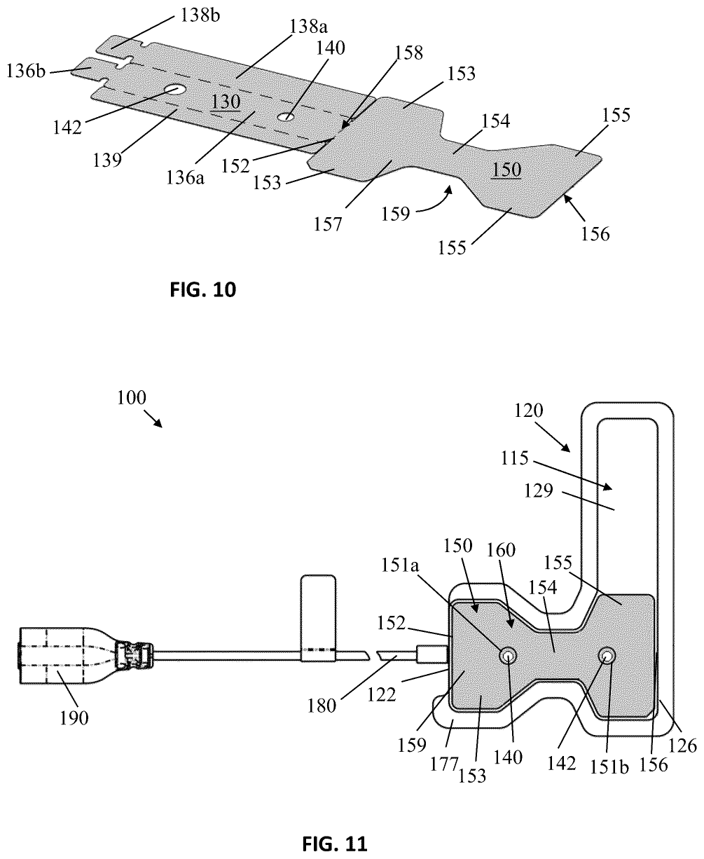

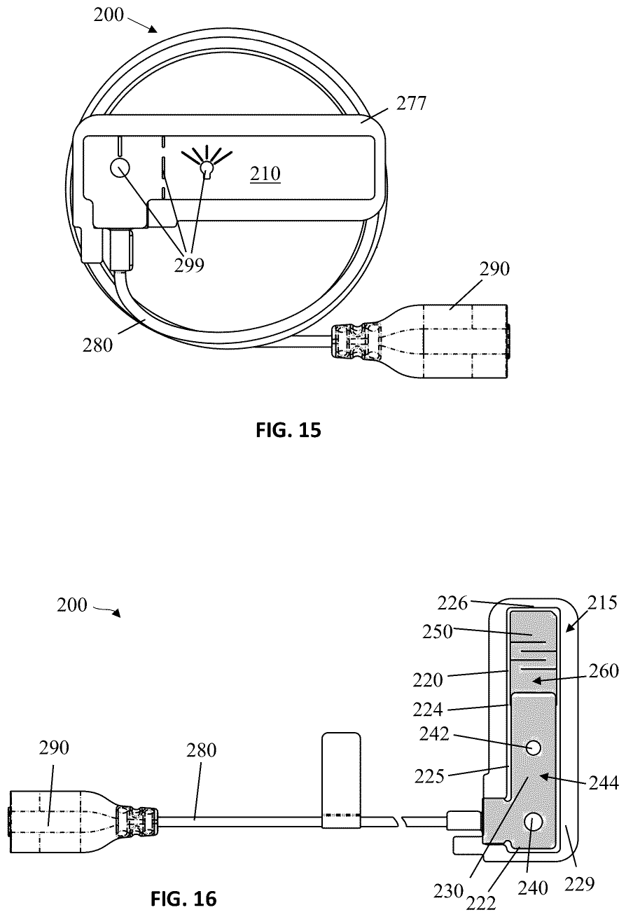

Referring now to FIGS. 14-24, sensory assembly 210 according to certain preferred aspects is illustrated. As shown in FIGS. 14 and 18, sensor assembly 210 is configured to be applied to tissue area 505 of patient 500. Sensor assembly 210 preferably includes substrate body 220, envelope assembly 230, and transmission assembly 270 having at least one light emitter 272 and at least one light detector 274. In some preferred aspects, as shown in FIG. 14, sensory assembly 210 is configured to be applied to a digit, such as a fingertip or toe, of patient 500. In some other preferred aspects, as shown FIG. 18, sensory assembly 210 is configured to be applied to other tissue areas of patient 500, such as a foot. Other tissue areas of a patient are contemplated, including an earlobe, palm of hand, or other body part that allows proper operable engagement of at least one light emitter 272 with at least one light detector 274 when applied to patient 500. While the sensor assembly 210 in FIGS. 14-24 is preferably configured for application to an infant or neonate, it is contemplated that sensor assembly 210 can also be applied to an adult or pediatric patient.

Sensor assembly 210 may be a “transmission type” type sensor, which is configured to include at least one light emitter 272 and at least one light detector 274 positioned on opposing sides of the sensor site of tissue area 505 and configured to operably engage with each other, whether the sensor site is a digit, such as a toe as shown in FIG. 14, or another body part, such as a foot as shown in FIG. 18. During operation, light emitter 272 transmits one or more wavelengths of light through the tissue area 505, and the light received by the light detector 274 is processed to determine various physiological characteristics of the patient, such as SpO₂. In the disclosure herein, it should be understood that the relative locations of the light emitter 272 and light detector 274 may be exchanged without substantially departing from the spirit of the disclosure.

Substrate body 220 includes intermediate portion 224 extending between first end 222 and opposing second end 226. In some preferred aspects, width of substrate body 220 is approximately the same the entire length of substrate body. In some other preferred aspects, a portion of substrate body 220 is wider located proximate first end 222 than second end 226.

In some preferred aspects, width of substrate body 220 is between about 0.35 inches and about 0.70 inches, in some aspects between about 0.40 inches and about 0.65 inches, and in some preferred aspects between about 0.45 inches and about 0.60 inches. In some other preferred aspects, width of substrate body 220 is between about 0.40 inches and about 1.0 inches, in some aspects between about 0.5 inches and about 0.9 inches, and in some preferred aspects between about 0.65 inches and about 0.8 inches.

In some preferred aspects, substrate body 220 has an axial length between about 2 inches and about 6 inches, in some aspects between about 2.25 inches and about 5.5 inches, and in some preferred aspects between about 2.5 inches and about 4.5 inches.

Substrate body 220 preferably comprises a material as previously disclosed regarding substrate body 120.

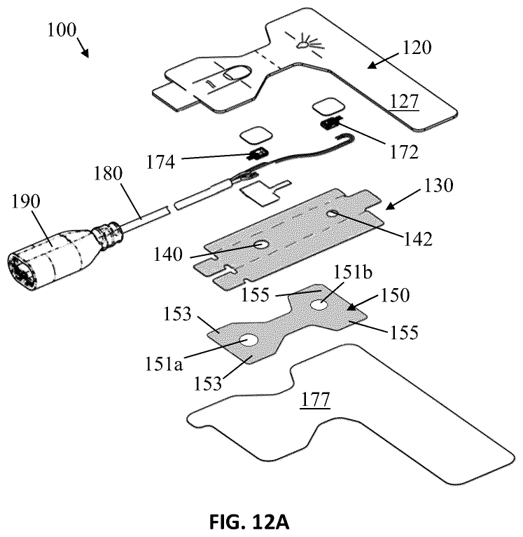

Substrate body 220 preferably includes first surface 227 configured to be located away from the tissue area 505 when sensor assembly 210 is applied to patient 500. In some preferred aspects, first surface 227 is substantially free of any adhesive material. As shown in FIGS. 15, 17, 19 and 21, first surface 227 may have one or more indicia 299 related to proper placement of sensor assembly 110 on tissue area 505 of patient 500. For example, first surface 227 may have indicia 299 related to the fold line of sensory assembly 210, digit (e.g., finger or toe) placement, light emitter location, light detector location, or a combination thereof. In some preferred aspects, the one or more indicia 299 provide guidance for proper placement such that there is proper operable engagement of light emitter 272 and light detector 274 when sensor assembly 210 is applied to patient 500.

Substrate body 220 preferably includes second surface 229 configured to be facing towards tissue area 505 when sensor assembly 210 is applied to patient 500. In some preferred aspects, second surface 229 contains an adhesive layer 215. In some preferred aspects, at least a portion of adhesive layer 215 is in direct contact with tissue area 505 when sensor assembly 110 is applied to patient 500. In some preferred aspects, adhesive layer 215 comprises a biocompatible material.

Envelope assembly 230 is preferably disposed relative to second surface 229 of substrate body 220. In some preferred aspects, at least a portion of adhesive layer 215 is located between second surface 229 and envelope assembly 230. Envelope assembly 230 is preferably disposed relative to second surface 229 such that a proximal end 232 is located proximate first end 222 of substrate body 220 and a distal end 234 is located proximate between second end 226 and intermediate portion 224 of substrate body 220. In some preferred aspects, envelope assembly 230 has a length that is less than the axial length of substrate body 220. In some preferred aspects, envelope assembly 230 has a width that is equal to or less than the width of substrate body 220 located proximate proximal end 232 and/or intermediate portion 224.

In some preferred aspects, envelope assembly 230 has an axial length between about 1.0 inches and about 3.0 inches, in some aspects between about 1.25 inches and about 2.5 inches, and in some other preferred aspects between about 1.5 inches and about 2.0 inches. In some preferred aspects, envelope assembly 230 has a width between about 0.35 inches and about 0.70 inches, in some aspects between about 0.40 inches and about 0.60 inches, and in some preferred aspects between about 0.45 inches and about 0.55 inches.

Envelope assembly 230 preferably comprises a first layer 236 and an opposing second layer 238. Second layer 238 can be configured to be located between second surface 229 of substrate body 220 and first layer 236, such that first layer 236 can be located closer to tissue area 505 than second layer 238. In some preferred aspects, at least a portion of adhesive layer 215 is in direct contact with second layer 238.

First layer 236 preferably has a first aperture 240 spaced apart a distance from a second aperture 242. In some preferred aspects, first aperture 240 is located between first end 222 and intermediate portion 224 of substrate body. In some preferred aspects, second aperture 242 is located proximate intermediate portion 224 of substrate body. The distance between first and second apertures 240, 242 is preferably between about 0.70 inches and 1.5 inches, in some aspects between 0.75 inches and about 1.2 inches, and in some preferred aspects between about 0.80 inches and about 1.1 inches.

In some preferred aspects, first aperture 240 has a radius that is preferably larger in size than the radius of second aperture 242.

First and second layers 236, 238 can be separate layers. When comprised of separate layers, first and second layers 236, 238 can be configured to be at least partially adhered together to form envelope assembly 230. In some aspects, an adhesive is applied between at least a portion of first and second layers 236, 238.

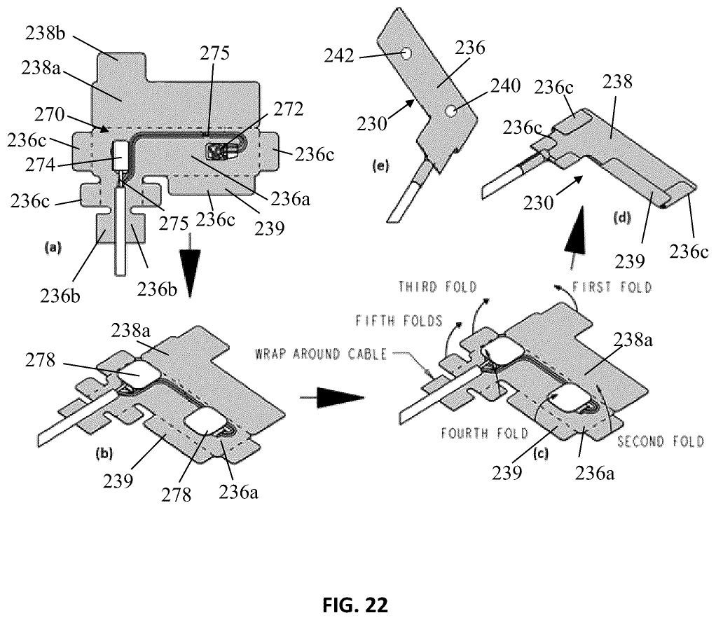

Envelope assembly 230 is preferably configured from a single sheet of material in a folded configuration to form first and second layers 236, 238. For instance, as shown in FIG. 22, envelope assembly 230 prior to the folded configuration can have two or more sections. In some preferred aspects, envelope assembly 230 prior to the folded configuration has at least three sections, whereby a first section 236a forms at least a portion of first layer 236, a second section 238a folded relative to first section 236a forms at least a portion of second layer 238, and a third section 239 folded relative to first section 236a operably engages with second section 238a to form second layer 238. In some aspects, first section 236a has an L-shaped configured. First section 236a can include one or more proximal member portions 236b that can operably engage with a portion of cable. First section 236a preferably includes two opposing member portions 236b that can operably engage with a portion of cable. In some aspects, second first section 238a can include one or more proximal member portions 238b that can operably engage with a portion of first section 236a. In some aspects, first section 236a can include one or more distal member portions 236c that can be folded to operably engage with at least a portion of second section 238a. First section 236a preferably includes two or more distal member portions 236c that can be folded to operably engage with different portions of second section 238a, such as shown in step (d) of FIG. 22.

Envelope assembly 230 preferably includes at least one layer that comprises a light absorption material. In some aspects, first layer 236 or second layer 238 comprises a light absorption material. In some preferred aspects, as shown best in FIG. 22, both first layer 236 and second layer 238 of envelope assembly 230 comprise a light absorption material.

Transmission sensor assembly 270 is preferably at least partially contained between first and second layers 236, 238 of envelope assembly 230. Transmission sensor includes at least one light emitter 272 and at least one light detector 274, whereby each of light emitter 272 and light detector 274 are located proximate first and second apertures 240, 242. Light emitter 272 is preferably located proximate first aperture 240, and light detector 274 is preferably located proximate second aperture 242. In some alternative aspects, light emitter 272 is preferably located proximate second aperture 242, and light detector 274 is preferably located proximate first aperture 240. Light emitter 272 is configured to emit at least one wavelength of light into the tissue area 505 of the patient, and light detector 274 is configured to detect at least one wavelength that has been emitted by light emitter 272, transmitted through the tissue area 505 of the patient and received by light detector 274. In some aspects, the at least one wavelength of light emitted by light emitter 272 is a range of wavelengths, and the at least one wavelength received by light detector 274 is the range of wavelengths emitted from light emitter 272. In some preferred aspects, light emitter 272 is configured to emit a first wavelength and a second wavelength into the tissue area 505 of the patient, and light detector 274 is configured to detect the first and second wavelengths that has been emitted by light emitter 272, transmitted through the tissue area 505 of the patient, and received by light detector 274.

In some preferred aspects, the first wavelength of light emitted by light emitter 272 is in the range between 620 nm and about 750 nm, more preferably between 630 nm and 690 nm, and even more preferably between 650 nm and 670 nm. In some preferred aspects, the first wavelength of light is at about 660 nm.

In some preferred aspects, the second wavelength of light emitted by light emitter 272 is in the range between 780 nm and about 1200, more preferably between 880 nm and about 940 nm, and even more preferably between 900 nm and 920 nm. In some preferred aspects, the second wavelength of light is at about 910 nm.

In some aspects, when sensor assembly 210 is properly applied to tissue area 505 of the patient 500, light detector 274 and light emitter 272 are configured to be positioned on opposing sides of tissue area 505, such that light detector 274 is configured to operably engage with light emitter 272 to detect the at least the first and second wavelengths of light emitted by light emitter 272 and transmitted through the sensor site of patient 500, and light detector 274 can be configured to generate at least one electrical signal relating to the at least one physiological parameter of patient 500. In some preferred aspects, the at least one signal relates to an SpO₂ measurement of the patient. In some preferred aspects, the at least one signal relates to an SpO₂ measurement and pulse rate of the patient. When sensor assembly 210 is properly applied to tissue area 505 of the patient 500, light detector 274 is preferably configured to be substantially aligned with light emitter 272 for the detection of the at least first and second wavelengths of light emitted by light emitter 272 and transmitted through the sensor site the patient. The alignment between light emitter 272 and light detector 274 may include some degree of offset without substantially affecting the detection of the at least the first and second wavelengths of light emitted by light emitter 272 and transmitted through the sensor site of patient 500.

Sensor assembly 210 can preferably include a light absorption layer 250. Light absorption layer 250 preferably comprises a light absorption material. Light absorption layer 250 preferably has a shaped configuration that is substantially similar to the shaped configuration of substrate body 220. In some exemplary aspects, light absorption layer 250 has a generally square, oval, rectangular, or other oblong shaped configuration.

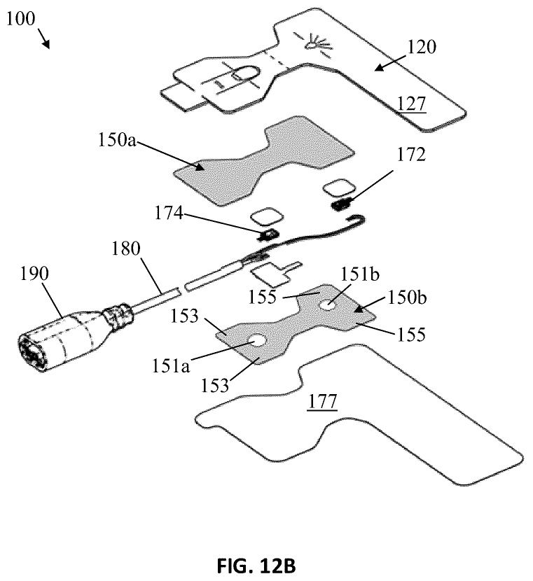

In some preferred aspects, as shown in FIGS. 16-17 and 20-21, light absorption layer 250 includes intermediate portion 254 extending between first end 252 and opposing second end 256. Light absorption layer 250 includes first surface 257 that operably engages with substrate body 220 and second surface 229 that is configured for operable engagement with tissue area 505 of patient 500. In some preferred aspects, at least a portion of adhesive layer 215 is located between second surface 229 of substrate body 220 and first surface 257 of light absorption layer 250.

First end 252 of light absorption layer 250 is preferably operably connected to distal end 234 of envelope assembly 230 located proximate intermediate portion 224 of substrate body 220. First end of light absorption layer 250 is preferably operably connected to distal end 234 of envelope assembly 230 with an adhesive 244, which can be located on second layer 238 or first layer 236 of envelope assembly 230. Second end 256 of light absorption layer 250 preferably is located proximate second end 226 of substrate body 220.

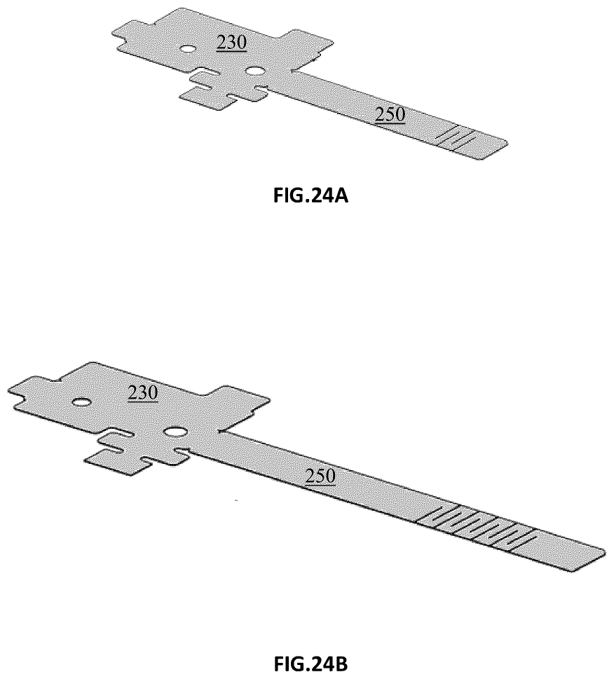

In some aspects as shown in FIGS. 24A-24B, envelope assembly 230 and light absorption layer 250 can be formed from a single sheet of material prior to a folded configuration, such that the envelope assembly 230 and light absorption layer 250 are combined into one folded configuration. In some preferred aspects, in the unfolded configuration, light absorption layer 250 extends from one of the distal member 236c positions of the envelope assembly 230. In some preferred aspects, in the unfolded configuration, light absorption layer 250 extends from the distal member 236c position located closest to proximal tab portions 236b. In some preferred aspects, light absorption layer 250 extends away from envelope assembly 230 in a configuration such that first and second apertures 240, 242 and light absorption layer 250 share a common axial line.

In some exemplary aspects, light absorption layer 250 has a surface area that substantially overlaps at least a portion of substrate body 220 with the exception of the portion of substrate body 220 having envelope assembly 210. In some exemplary aspects, second surface 229 has a surface area that is slightly larger than the surface area of the portion of light absorption layer 250 that is operably engaged with second surface 229. In some other exemplary aspects, the surface area of light absorption layer 250 relative to the surface area of second surface 229 is slightly smaller, such that there is a gap between the perimeter of light absorption layer 250 and perimeter of the second surface 229 that is less than 2 mm, in some aspects less than 1.5 mm, and in some other aspects less than 1 mm. In some other exemplary aspects, the surface area of envelope assembly 230 relative to the surface area of second surface 229 is slightly smaller, such that there is a gap between the perimeter of envelope assembly 230 and perimeter of the second surface 229 that is less than 2 mm, in some aspects less than 1.5 mm, and in some other aspects less than 1 mm.

In some preferred aspects, light absorption layer 250 has a width (W) between about 0.35 inches and about 0.70 inches, in some aspects between about 0.40 inches and about 0.60 inches, and in some preferred aspects between about 0.45 inches and about 0.55 inches. Light absorption layer 250 preferably has an axial length (Lₐ) between about 0.75 inches and about 3.0 inches, in some aspects between about 0.8 inches and about 2.75 inches, and in some other preferred aspects between about 1.0 inches and about 2.5 inches.

In some preferred aspects, light absorption layer 250 has an uncut portion between one end and the first slit 255 having a length (Lᵤ) that is between about 0.25 and about 0.80 inches, in some aspects between about 0.30 and about 0.75 inches, and in some preferred aspects between about 0.25 and about 0.70 inches.

Light absorption layer 250 preferably can have adhesive layer 260 provided on second surface 229 for applying sensor assembly 210 to the tissue area 505 of the patient 500 to maintain proper placement of sensor assembly 210 on the patient and operable engagement of light emitter 272 and light detector 274 during non-motion and motion activities. In some aspects, light absorption layer 250 can have an adhesive layer 260 also provided on first surface 257, such that adhesive layer 260 provided on first surface 257 of light absorption layer 250 operably engages adhesive layer 215 of substrate body 220 and/or a portion of adhesive layer 244 of envelope assembly 230.

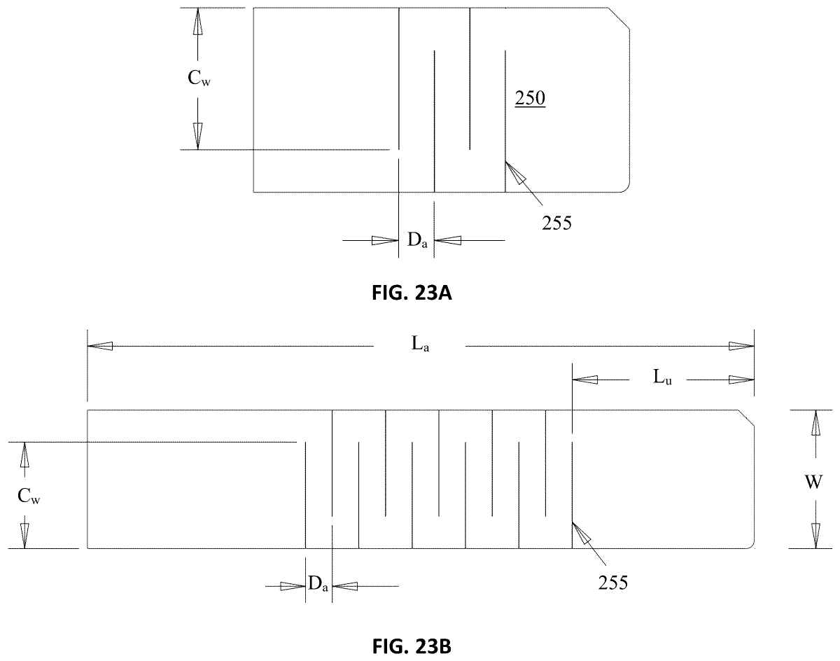

Light absorption layer 250 may have one or more slits 255 located proximate intermediate portion 254. Light absorption layer 250 preferably has a plurality of slits 255 located proximate intermediate portion 254. Light absorption layer 250 preferably has at least 3 and up to about 24 slits, preferably between 3 and 15, and more preferably between 3 and 12 slits, located proximate intermediate portion 254. Slits 255 are preferably cut through a portion of light absorption layer 250 from at least one side. In some preferred aspects, slits 255 are cut from opposing sides of light absorption layer 250 in an alternating arrangement, such as shown in FIGS. 23A-23B. In some preferred aspects, the distance (Dₐ) between each adjacent slit 255 is about 0.05 to about 0.3 inches, in some aspects between 0.75 and 0.2 inches, and in some aspects between 0.8 and 0.15 inches. Each slit 255 preferably is cut (C_{w}) through more than half the width of light absorption layer 250. In some preferred aspects, one or more slits 255 are cut part way through the width (C_{w}) of the light absorption layer 250 a distance between 0.25 inches and about 0.5 inches, in some aspects between about 0.30 inches and about 0.49 inches, and in some preferred aspects between about 0.4 inches and about 0.48 inches.

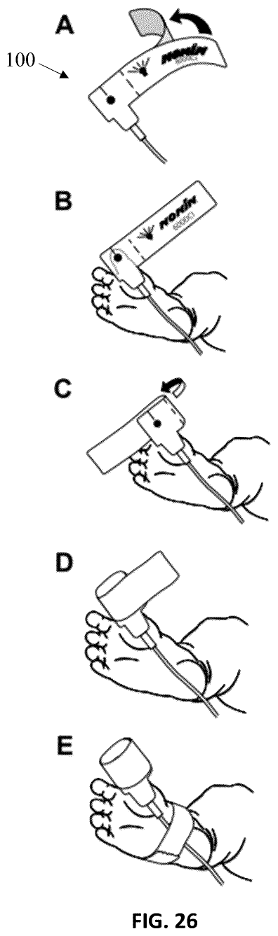

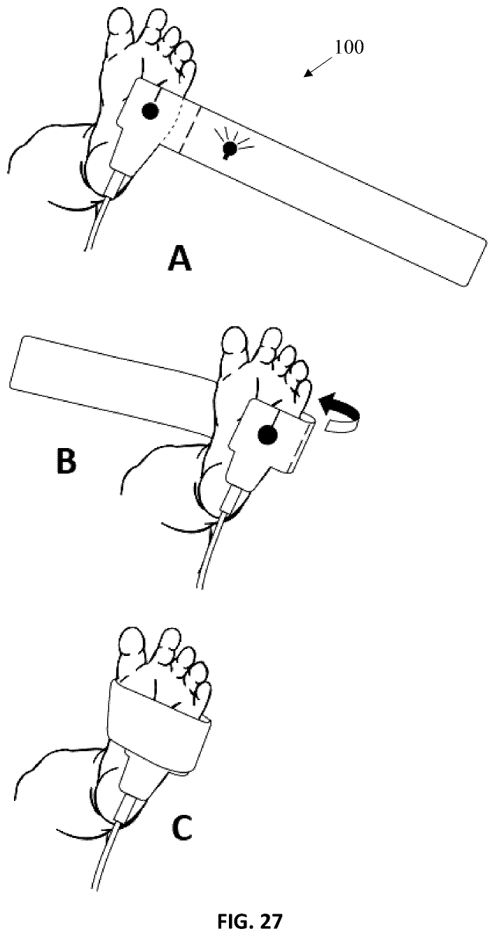

Each sensor assembly 210 of the foregoing exemplary disclosure included substrate body 220 operably engaged with envelope assembly 230 and light absorption layer 250, wherein envelope assembly 230 and light absorption layer 250 had minimal, if any, overlap. Referring now to FIGS. 26 and 27 in some exemplary aspects, sensor assembly 210 is applied to tissue area 505 of patient 500. The application of sensor assembly 210 preferably includes applying first end 222 of substrate body 210 to tissue area 505 of patient 500 prior to second end 226 is applied. Sensor assembly 210 may contain release liner 277 that operably engages patient engagement adhesive of sensor assembly 210. Release liner 277 can be partially or completely peeled away from sensor assembly 210, as shown in steps (A) of FIGS. 26 and 27. The tissue area 505 of patient 500, shown as a toe in FIG. 26 and a foot in FIG. 27, can be placed with respect to the tissue engagement surface of sensor assembly 210. In some exemplary aspects relating to a digit (e.g., finger or toe), first end 226 having light detector 274 is located on the bottom of the digit. In some exemplary aspects relating to a foot or hand, first end 226 having light detector 274 is located on the bottom of the foot or hand. If the release liner 277 has not been completely removed, the release liner 277 can be completely removed. The remaining portion of sensor assembly 210, which includes the light absorption layer 250 and second end 226 is wrapped around the tissue area 505, such as around the digit or around the foot/hand, while ensuring light emitter 272 is directly opposite light detector 274 for proper operable engagement, such as shown in steps (B-E) in FIG. 26 and steps (B-C) in FIG. 27. In some preferred aspects, light emitter 272 and light detector 274 are engaged for operable engagement, such as by use of one or more indicia. In some preferred aspects, the remaining portion of sensor assembly 210 is wrapped around tissue area 505, such that second end 226 overlaps a portion of substrate body 220, preferably being adhered with a portion of first surface 127 of substrate body 220, which in some aspects may be first end 222 or intermediate portion 224.

In some aspects, tape such as medical tape can be applied to a portion of cable assembly 280 independently from sensor assembly 210 to ensure the integrity of proper operable engagement of light emitter 272 and light detector 274 is maintained during normal operational use.

The light absorption material of envelope assembly 130, 230, light absorption layer 150, 250, and/or substrate body 120, may be a substantially dark color, such as black. In some aspects, the light absorption material has a matte finish. In some aspects, the light absorption material has a matte black surface. In some preferred aspects, the light absorption material is a thermal laminate film. The thermal laminate film preferably has an adhesive on at least one side. In some exemplary aspects, the thermal laminate film is a black thermal laminating film comprising a polypropylene base/ethylene-vinyl acetate, such as a polyamide base/blended ethylene-vinyl acetate and polyol, provided by Skandacor™ as SOFTpro Black Laminating Film. The thermal laminate film preferably has a lamination tensile strength greater than 1.7 N/25 mm in relation to the biaxially oriented polypropylene side of the thermal laminate film. The thermal laminate film preferably has a lamination tensile strength greater than or equal to 7.5 N/25 mm on the adhesive side. In some other aspects, the light absorption layer can be a polyester film having a tackified acrylic adhesive.

The light absorption material is preferably flexible and/or malleable, such as allowing the envelope assembly 130 and/or light absorption layer 150 to conform to the tissue area of the patient. In certain aspects, each layer of the light absorption material has a thickness between about 0.5 mils and about 3.0 mils, in some aspects between about 0.8 mils and about 2.0 mils, and in some preferred aspects between about 1.0 mils and 1.5 mils. In some preferred aspects, the light absorption material of envelope assembly 130, 230 and/or light absorption layer 150, 250 has a substantially uniform thickness.

Without wishing to be bound by theory, a light absorption material, such as that located on the nonengagement side of transmission assembly 170, 270, can at least partially block ambient light, such that external light from an external light source other than light emitter 172, 272 is not sensed by light detector 174, 274. As provided by the foregoing disclosure, the light absorption material may be located in different locations to at least partially block ambient light, including on the first surface 127, 227 of substrate body 120, 220, as at least part of substrate body 120, 220, between second surface 129, 229 of substrate body 120, 220 and envelope assembly 130, 230 as a separate light absorption layer 150, 250, as at least part of second surface 138, 238 of envelope assembly 130, 230, or a combination thereof.

Without wishing to be bound by theory, a light absorption material, such as that located on the engagement side of transmission assembly 170, 270 can at least partially block shunted light, such that light from light emitter 172, 272 that does not pass through tissue 505 of patient 500 is not sensed by light detector 174, 274. As provided by the foregoing disclosure, the light absorption material may be located in different locations to at least partially block shunted light, including as at least part of first surface 136, 236 of envelope assembly 130, 230, as a separate light absorption layer 150, 250 located between transmission assembly 170, 270 and tissue 505 of patient 500, or a combination thereof.

In some preferred aspects, the light absorption material absorbs at least 80%, in some aspects at least 85%, in some aspects at least 90%, in some aspects at least 95%, and in some aspects at least 99% of at least one wavelength of shunted light transmitted by light emitter 172, 272 that contacts the light absorption material. In some preferred aspects, the light absorption material absorbs at least 80%, in some aspects at least 85%, in some aspects at least 90%, in some aspects at least 95%, and in some aspects at least 99% of a first wavelength between 620 nm and about 750 nm, more preferably between 630 nm and 690 nm, and even more preferably between 650 nm and 670 nm, of shunted light transmitted by light emitter 172, 272 that contacts the light absorption material.

In some preferred aspects, the light absorption material absorbs less than 99%, in some aspects less than 95%, in some aspects less than 90%, and in some aspects less than 85% of a second wavelength between 780 nm and about 1200, more preferably between 880 nm and about 940 nm, and even more preferably between 900 nm and 920 nm, of shunted light transmitted by light emitter 172, 272 that contacts the light absorption material.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 70-100% for skin pigmentation types of (i) light skin pigmentation, (ii) intermediate skin pigmentation, and/or (iii) dark skin pigmentation, wherein the skin pigmentation type as preferably determined by ITA skin color type, Monk Skin Tone Scale, and/or Fitzpatrick skin color type.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 70-80% for light skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 70-80% for intermediate skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 70-80% for dark skin pigmentation.

In some preferred aspect, the sensor assembly 110, 210 is configured to provide an A_{RMS} value less than 3.0, in some aspects less than 2.5, in some aspects less than 2.4, in some aspects less than 2.3, in some aspects less than 2.2, in some aspects less than 2.1, and in some preferred aspects less than 2.0, measured across the saturation range of 80-90% for skin pigmentation types of (i) light skin pigmentation, (ii) intermediate skin pigmentation, and/or (iii) dark skin pigmentation, wherein the skin pigmentation type is determined, for example, by ITA skin color type, Monk Skin Tone Scale and/or Fitzpatrick skin color type.

In some preferred aspect, the sensor assembly 110, 210 is configured to provide an A_{RMS} value less than 3.0, in some aspects less than 2.5, in some aspects less than 2.4, in some aspects less than 2.3, in some aspects less than 2.2, in some aspects less than 2.1, and in some preferred aspects less than 2.0, measured across the saturation range of 80-90% for light skin pigmentation.

In some preferred aspect, the sensor assembly 110, 210 is configured to provide an A_{RMS} value less than 3.0, in some aspects less than 2.5, in some aspects less than 2.4, in some aspects less than 2.3, in some aspects less than 2.2, in some aspects less than 2.1, and in some preferred aspects less than 2.0, measured across the saturation range of 80-90% for intermediate skin pigmentation.

In some preferred aspect, the sensor assembly 110, 210 is configured to provide an A_{RMS} value less than 3.0, in some aspects less than 2.5, in some aspects less than 2.4, in some aspects less than 2.3, in some aspects less than 2.2, in some aspects less than 2.1, and in some preferred aspects less than 2.0, measured across the saturation range of 80-90% for dark skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 90-100% for skin pigmentation types of (i) light skin pigmentation, (ii) intermediate skin pigmentation, and/or (iii) dark skin pigmentation, wherein the skin pigmentation type as preferably determined by ITA skin color type, Monk Skin Tone Scale, and/or Fitzpatrick skin color type.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 90-100% for light skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 90-100% for intermediate skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 90-100% for dark skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 88-92% for skin pigmentation types of (i) light skin pigmentation, (ii) intermediate skin pigmentation, and/or (iii) dark skin pigmentation, wherein the skin pigmentation type as preferably determined by ITA skin color type, Monk Skin Tone Scale, and/or Fitzpatrick skin color type.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 88-92% for light skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 88-92% for intermediate skin pigmentation.

In some preferred aspect, the sensor assembly 110, 210 is configured to provide an A_{RMS} value less than 2.5, in some aspects less than 2.4, in some aspects less than 2.3, in some aspects less than 2.2, in some aspects less than 2.1, in some aspects less than 2.0, and in some preferred aspects less than 1.9, measured across the saturation range of 88-92% for dark skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 70-100% for skin pigmentation types of (i) light skin pigmentation, (ii) intermediate skin pigmentation, and/or (iii) dark skin pigmentation, wherein the skin pigmentation type is determined, for example, by ITA skin color type, Monk Skin Tone Scale and/or Fitzpatrick skin color type.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 70-80% for skin pigmentation types of light skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 70-80% for skin pigmentation types of intermediate skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 70-80% for skin pigmentation types of dark skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 80-90% for skin pigmentation types of light skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 80-90% for skin pigmentation types of intermediate skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 80-90% for skin pigmentation types of dark skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 90-100% for skin pigmentation types of light skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 90-100% for skin pigmentation types of intermediate skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 90-100% for skin pigmentation types of dark skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 88-92% for skin pigmentation types of light skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 88-92% for skin pigmentation types of intermediate skin pigmentation.

In some preferred aspect, the sensor assembly 110, 210 is configured to provide an overall bias of less than 1.5, in some aspects less than 1.4, in some aspects less than 1.3, in some aspects less than 1.2, in some aspects less than 1.1, in some aspects less than 1.0, in some aspects less than 0.9, in some aspects less than 0.8, in some aspects less than 0.7, in some aspects less than 0.6, in some aspects less than 0.5, in some aspects less than 0.4, in some aspects less than 0.3, and in some preferred aspects less than 0.2, measured across the saturation range of 88-92% for skin pigmentation types of dark skin pigmentation.

Referring now to FIGS. 4, 6, 8, 12, 17, 21 and 22, transmission assembly 170, 270 of sensor assembly 110, 210 may include one or more respective lead wires 175, 275 that are operably connected with cable assembly 180, 280. In exemplary aspects, one or more lead wires 175 is electrically connected to light emitter 172, 272 and one or more different lead wires 175, 275 is electrically connected to light detector 174, 274. Transmission assembly 170, 270 may include a copper shield 176, 276 operably engaged with light detector 174, 274 to top side of one or more lead wires 175, 275 to maximize detection and minimize electrical crosstalk between those component aspects. Transmission assembly 170, 270 may include one or more light blocker materials 178, 278 applied to the opposite side of the light emitting side of light emitter 172, 272 and/or the light receiving side of light detector 174, 274, further minimizing electrical crosstalk between those component aspects. In some preferred aspects, light blocker material 178, 278 is applied on top of copper shield 176, 276 with respect to light detector 174, 274.

Cable assembly 180, 280 preferably includes one or more wires configured for operable connection of sensor assembly 110, 210 with connector assembly 190, 290. In some preferred aspects, cable assembly 180, 280 includes at least one wire configured for operable connection with light emitter 172, 272. In some other preferred aspects, cable assembly 180, 280 includes two or more wires for operable connection with light emitter 180, 280, a first wire configured to facilitate the generation of a first wavelength, and second wire configured to facilitate the generation of a second wavelength. In some preferred aspects, cable assembly 180, 280 includes at least one wire configured for operable connection with light detector 174, 274. In some other preferred aspects, cable assembly 180, 280 includes two or more wires for operable connection with light detector 174, 274.

Connector assembly 190, 290 is preferably configured for operable connection of sensor assembly 110, 210 with a pulse oximetry monitor (not shown). Monitor may be any suitable pulse oximeter, including monitors that provide continuous or periodic monitoring. In some preferred aspects, SpO₂ measurements can be displayed on monitor. In some other aspects, monitor is a multi-parameter patient monitor capable of monitoring SpO₂ measurements, and in some aspects one or more other physiological parameters besides SpO₂.

In some preferred aspects, connector assembly 190, 290 is an intermediate connection to sensor assembly 110, 210. In some preferred aspects, connector assembly 190, 290 has an adapter for operable connection with another component. The adapter can have different configurations for appropriate connection with another component. In some aspects, adapter is configured to be operably connected with another cable to facilitate an electrical transmission between sensor assembly 110, 210 and the monitor. In some aspects, the electrical transmission between sensor assembly 110, 210 and the monitor comprises a signal generated in relation to the measurement of SpO₂ of a patient by sensor assembly 110, 210. In some other aspects, the adapter is configured to be operably connected with a transmission device to facilitate a wireless transmission between sensor assembly 110, 210 and the monitor. In some aspects, the wireless transmission between device transmits a signal generated by sensor assembly 110, 210 to the monitor in relation to the measurement of a physiological parameter, such as the measurement of SpO₂ of a patient.

### Example

A performance assessment of non-invasive pulse oximetry sensors of the present disclosure was conducted according to the basic pulse oximeter hypoxia (lab) performance study protocol designed by the UCSF Hypoxia Lab that was the original model for the FDA and later ISO80601-2-61 requirements and standards for pulse oximetry performance. The present performance assessment was conducted by monitoring SpO₂ measurements of twenty-six (26) healthy adult (ages 18-50) patient subjects having skin pigmentation types of either light skin pigmentation, intermediate skin pigmentation, or dark skin pigmentation.

The skin pigmentation type of each of the twenty-six (26) patient subjects was determined by questions asked of the subjects for evaluation of Fitzpatrick skin type and also by measurements using a Konica Minolta CM-700d to determine the ITA skin color type. The Fitzpatrick levels resulted in three (3) patient subjects having Fitzpatrick level I, seven (7) patient subjects having Fitzpatrick level II, three (3) patient subjects having Fitzpatrick level III, six (6) patient subjects having Fitzpatrick level IV, four (4) patient subjects having Fitzpatrick level V, and three (3) patient subjects having Fitzpatrick level VI. Based upon the Fitzpatrick level and the ITA skin color type, there were ten (10) patient subjects (38%) assessed as having light skin pigmentation, nine (9) patient subjects (35%) assessed as having intermediate skin pigmentation, and seven (7) patient subjects (27%) assessed as having dark skin pigmentation.

Patient monitoring sensor No. 1 (Sensor No. 1) is generally shown in FIGS. 1-4 with the envelope assembly, except envelope assembly of Sensor No. 1 did not have any light absorption material. Patient monitoring sensor No. 2 (Sensor No. 2) is generally shown in FIGS. 1-4 with the envelope assembly having the light absorption material for both the first and second layers of the envelope assembly. Patient monitoring sensor No. 3 (Sensor No. 3) is generally shown in FIGS. 7-8 with the envelope assembly and the light absorption layer having the light absorption material for both the first and second layers of the envelope assembly and the light absorption layer. Each of Sensor Nos. 1-3 were applied to the index finger of the subject patient consistent with the disclosure relating to FIG. 25, such that the light emitter and light detector were in proper operable engagement for a transmittance type sensor for both non-motion hand conditions and motion hand conditions.

Light emitter emitted red and infrared light, and the light detector detected the red wavelength emitted at 660 nanometers at 3 mW nominal and the infrared wavelength at 910 nanometers at 3 nW nominal.

Each subject patient had an arterial catheter inserted. Each subject patient was subjected to controlled hypoxemia during non-motion conditions and also during motion conditions. During each of the non-motion and motion conditions, performance assessment was conducted over two (2) desaturation runs having a total of nine (9) plateaus reached for a period of time of at least 20 seconds, but typically 60-92 seconds at each level. Between 2 and 4 arterial blood samples were obtained at each of the different steady-state levels of hypoxemia from 70-100% for a total of 30 arterial blood samples.

The overall A_{RMS} and mean bias performance assessment data for each of Sensor Nos. 1-3 in relation to non-motion conditions was assessed by SpO₂ ranges of 70-80, 80-90, 90-100 and also the overall range of 70-100. The non-motion performance assessment data for both A_{RMS} and mean bias were significantly better for each of Sensor Nos. 2 and 3 compared to Sensor No. 1.

Sensor No. 1 had an average A_{RMS} value of 2.9 for the overall SpO₂ range of 70-100, and an average A_{RMS} value over 4.0 for the SpO₂ range of 70-80. Sensor Nos. 2 and 3 had average A_{RMS} values less than 2.0 for the overall SpO₂ range of 70-100, and each of Sensor Nos. 2 and 3 also had (i) average A_{RMS} values of 2.2 for the SpO₂ range of 70-80, (ii) average A_{RMS} values of less than 2.0 for the SpO₂ range of 80-90, and (iii) average A_{RMS} values of 1.5 or less for the SpO₂ range of 90-100.

Sensor No. 1 had an average mean bias value greater than 1.5 for the overall SpO₂ range of 70-100, an average mean bias value greater than 3.5 for the SpO₂ range of 70-80, and an average mean bias value greater than 1.0 for the SpO₂ range of 80-90. Sensor Nos. 2 and 3 had average mean bias values less than 0.5 for the overall SpO₂ range of 70-100, and each of Sensor Nos. 2 and 3 also had (i) average mean bias values of 0.6 or less for the SpO₂ range of 70-80, (ii) average mean bias values of 0.3 or less for the SpO₂ range of 80-90, and (iii) average mean bias values of 0.3 or less for the SpO₂ range of 90-100. As compared with Sensor No. 2, Sensor No. 3 showed greater overall accuracy.

The overall A_{RMS} and mean bias performance assessment data for each of Sensor Nos. 2-3 in relation to motion conditions was also assessed for all adult patient population. The motion performance assessment data was comparable to the A_{RMS} and average mean bias for the non-motion conditions. Sensor Nos. 2 and 3 had average A_{RMS} values less than 2.0 for the overall SpO₂ range of 70-100. Each of Sensor Nos. 2 and 3 also had (i) average A_{RMS} values of 2.2 for the SpO₂ range of 70-80, (ii) average A_{RMS} values of 1.7 or less for the overall SpO₂ range of 80-90, and (iii) average A_{RMS} values of 1.6 or less for the overall SpO₂ range of 90-100. Sensor Nos. 2 and 3 also had average mean bias values less than 0.5 for the overall SpO₂ range of 70-100. Each of Sensor Nos. 2 and 3 also had (i) average mean bias values of 0.6 or less for the SpO₂ range of 70-80, (ii) average mean bias values of 0.2 or less for the SpO₂ range of 80-90, and (iii) average mean bias values of 0.5 or less for the SpO₂ range of 90-100. As compared with Sensor No. 2, Sensor No. 3 showed greater overall accuracy, indicating that the configuration having both the envelope assembly and the light absorption layer with the light absorption material had better overall performance.

The overall mean bias performance assessment data for each of Sensor Nos. 2-3 in relation to non-motion and motion was assessed by overall SpO₂ ranges of 70-85 and also >85 and also by skin pigmentation types of (i) light skin pigmentation, (ii) intermediate skin pigmentation, and (iii) dark skin pigmentation, each at SpO₂ ranges of 70-85 and >85. The non-motion and motion performance assessment data for Sensor No. 3 provided a tighter data fit at saturations greater than 85% SpO₂ as compared to Sensor No. 2.

The skin pigmentation bias per FDA guidance between skin pigmentation types for each of Sensor Nos. 2-3 in relation to non-motion and motion conditions was assessed by overall SpO₂ ranges of 70-85 and also >85 and also by skin pigmentation types of (i) light skin pigmentation, (ii) intermediate skin pigmentation, and (iii) dark skin pigmentation, each at SpO₂ ranges of 70-85 and >85. The non-motion and motion performance assessment of Sensor Nos. 2 and 3 were compliant with guidance under FDA considerations for bias between skin pigmentation types of (i) light skin pigmentation, (ii) intermediate skin pigmentation, and (iii) dark skin pigmentation, including <3.5 bias for non-motion and motion at SaO2% of 70-85%, and <1.5 bias for non-motion and motion at SaO2%>85%.

A comparison of overall A_{RMS} and mean bias performance assessment data for each of Sensor No. 1 and Sensor No. 3 was also assessed for all adult patient population based upon SpO₂ ranges of 70-80, 80-90, 90-100 and also the overall range of 70-100 and also skin pigmentation types of (i) light skin pigmentation, (ii) intermediate skin pigmentation, and (iii) dark skin pigmentation, for each of these SpO₂ ranges. The assessment indicated that both A_{RMS} and mean bias were significantly more accurate for Sensor No. 3 having the light absorption material in each of the envelope assembly and light absorption layer compared to Sensor No. 1 that did not have any light absorption material.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A patient monitoring sensor configured to generate at least one electrical signal relating to at least one physiological parameter of the patient, comprising:
a conformable substrate body having a first end and an opposing second end, the conformable substrate body having a first surface configured to be located away from the patient when the device is applied to the patient, and the conformable substrate body having a second surface configured to be directed toward a tissue area of the patient when the device is applied to the patient;
an envelope assembly comprising a first layer and an opposing second layer, the first layer being configured to be located closer to the second surface of the conformable substrate body than the second layer, the second layer having a first aperture spaced apart a distance from a second aperture, and the envelope assembly having a first end and an opposing second end; and
a transmission sensor assembly located between the first and second layers of the envelope assembly, the transmission sensor assembly comprising at least one light emitter located proximate the first aperture and at least one light detector located proximate the second aperture, wherein the at least one light emitter configured to transmit at least a first wavelength and at least a second wavelength, and the at least one light detector configured to detect each of the at least first and second wavelengths, wherein the at least one light emitter and the at least one light detector configured to be positioned on opposing sides of the tissue area of the patient when the device is applied to the patient, such that the light detector is configured to operably engage with the light emitter to detect the at least first and second wavelengths transmitted by the at least one light emitter through at least a portion of the tissue area of the patient;
wherein at least the first and second layers of the envelope assembly comprising a light absorption material configured to reduce or prevent the at least one light detector from detecting the at least first wavelength that does not pass through an intended light path through the portion of the tissue area of the patient from the at least one light emitter.

2. The patient monitoring sensor of claim 1, wherein the envelope assembly comprises a single sheet of light absorption material in a folded configuration forming the first and second layers.

3. The patient monitoring sensor of claim 1, wherein the first and second layers of the envelope assembly are formed of separate sheets of the light absorption material.

4. The patient monitoring sensor of claim 1, further comprising a light absorption layer comprising a light absorption material configured to reduce or prevent the at least one light detector from detecting the at least first wavelength that does not pass through an intended light path through the portion of the tissue area of the patient from the at least one light emitter, wherein the light absorption layer having a third aperture and a fourth aperture, wherein the envelope assembly is provided between the conformable substrate body and the light absorption layer, such that the first aperture of the envelope assembly and the third aperture of the light absorption layer have a first aligned configuration and the second aperture of the envelope assembly and the fourth aperture of the light absorption layer have a second aligned configuration.

5. The patient monitoring sensor of claim 1, further comprising a light absorption layer comprising a light absorption material provided between the conformable substrate body and the envelope assembly to reduce or prevent the at least one light detector from detecting the at least first wavelength that does not pass through an intended light path through the portion of the tissue area of the patient from the at least one light emitter.

6. The patient monitoring sensor of claim 5, wherein the light absorption layer has a greater surface area than each of the first and second layers of the envelope assembly.

7. The patient monitoring sensor of claim 6, wherein the light absorption layer having a first pair of opposing tab members located proximate the first end of the conformable substrate body, and the light absorption layer having a second pair of tab members located proximate the second end of the conformable substrate body, wherein an intermediate section of the light absorption layer located between the first and second pair of tabs has a narrower width than each of the first and second pair of tabs.

8. The patient monitoring sensor of claim 7, wherein the conformable substrate body having a first pair of opposing tab members located proximate the first end, the conformable substrate body having a second pair of opposing tab members located proximate the second end, wherein the second pair of opposing tab members has at least one elongated tape member having a length that is greater than the opposing tab member, and wherein an intermediate section of the conformable substrate body located between the first and second ends has a width that is less than each of the first and second ends.

9. The patient monitoring sensor of claim 5, wherein the light absorption layer having a plurality of slits, wherein the plurality of slits are configured to reduce or prevent the at least one light detector from detecting the at least first wavelength that does not pass through an intended light path through the portion of the tissue area of the patient from the at least one light emitter.

10. The patient monitoring sensor of claim 1, wherein at least one surface of the conformable substrate body is configured to be in direct contact with a retention mechanism, and wherein the retention mechanism is configured to maintain the patient monitoring sensor in direct contact with the tissue area of the patient when applied to the patient.

11. The patient monitoring sensor of claim 1, further comprising a connector in electrical or optical communication with the transmission sensor assembly via at least one lead, wherein the electrical connector is configured to be connected to a monitor, and wherein the monitor is configured to receive the at least one signal generated by the transmission sensor assembly relating the at least one physiological parameter of the patient.

12. The patient monitoring sensor of claim 1, wherein the at least one light emitter is configured to the first wavelength between about 620 nm and about 750 nm and the second wavelength between about 780 nm and about 1200 nm.

13. The patient monitoring sensor of claim 1, wherein the second surface of the conformable substrate body having a first adhesive layer, wherein at least a portion of the first adhesive layer is configured to be in direct contact with at least portion of the tissue area of the patient when the device is applied to the patient.

14. A patient monitoring sensor configured to generate at least one electrical signal relating to at least one physiological parameter of the patient, comprising:
a conformable substrate body having a first end and an opposing second end, the conformable substrate body having a first surface configured to be located away from the patient when the device is applied to the patient, and the conformable substrate body having a second surface configured to be directed toward a tissue area of the patient when the device is applied to the patient;
an envelope assembly comprising at least one layer having a first aperture spaced apart a distance from a second aperture, wherein the envelope assembly having a first end and an opposing second end;
a light absorption layer comprising a light absorption material provided to at least partially engage with the second surface of the conformable substrate body; and
a transmission sensor assembly located between the at least one layer of the envelope assembly and the light absorption layer, the transmission sensor assembly comprising at least one light emitter located proximate the first aperture and at least one light detector located proximate the second aperture, wherein the at least one light emitter configured to transmit at least a first wavelength and at least a second wavelength, and the at least one light detector configured to detect each of the at least first and second wavelengths, wherein the at least one light emitter and the at least one light detector configured to be positioned on opposing sides of the tissue area of the patient when the device is applied to the patient, such that the light detector is configured to operably engage with the light emitter to detect the at least first and second wavelengths transmitted by the at least one light emitter through at least a portion of the tissue area of the patient;
wherein the light absorption layer is configured to reduce or prevent the at least one light detector from detecting the at least first wavelength that does not pass through an intended light path through the portion of the tissue area of the patient from the at least one light emitter.

15. The patient monitoring sensor of claim 14, wherein the envelope assembly comprises a first and an opposing second layer, the first layer configured to be located closer to the second surface of the conformable substrate body and the light absorption layer than the second layer, and wherein the transmission sensor assembly located between the first and second layers of the envelope assembly.

16. The patient monitoring sensor of claim 15, wherein the light absorption layer is provided at least partially between the second surface of the conformable substrate body and the first layer of the envelope assembly.

17. The patient monitoring sensor of claim 16, wherein each of the first and second layers of the envelope assembly comprising a light absorption material to reduce or prevent the at least one light detector from detecting the at least first wavelength that does not pass through an intended light path through the tissue area of the patient from the at least one light emitter.

18. The patient monitoring sensor of claim 17, wherein the envelope assembly comprising a single sheet of light absorption material in a folded configuration forming the first and second layers.

19. The patient monitoring sensor of claim 17, wherein the first and second layers of the envelope assembly are formed of separate sheets of the light absorption material.

20. The patient monitoring sensor of claim 15, wherein the light absorption layer having a third aperture and a fourth aperture, wherein the envelope assembly is provided between the conformable substrate body and the light absorption layer, such that the first aperture of the envelope assembly and the third aperture of the light absorption layer having a first aligned configuration located proximate the at least one light emitter and the second aperture of the envelope assembly and the fourth aperture of the light absorption layer having a second aligned configuration located proximate the at least one light detector.

21. The patient monitoring sensor of claim 14, wherein the light absorption layer has a greater surface area than the at least one layer of the envelope assembly.

22. The patient monitoring sensor of claim 14, wherein the light absorption layer having a first pair of opposing tab members located proximate the first end of the conformable substrate body, and the light absorption layer having a second pair of tab members located proximate the second end of the conformable substrate body, wherein an intermediate section of the light absorption layer located between the first and second pair of tabs has a narrower width than each of the first and second pair of tabs.

23. The patient monitoring sensor of claim 14, wherein the conformable substrate body having a first pair of opposing tab members located proximate the first end, the conformable substrate body having a second pair of opposing tab members located proximate the second end, wherein the second pair of opposing tab members has at least one elongated tape member having a length that is greater than the opposing tab member, and wherein an intermediate section of the conformable substrate body located between the first and second ends has a width that is less than each of the first and second ends.

24. The patient monitoring sensor of claim 14, wherein the second surface of the conformable substrate body having a first adhesive layer, wherein at least a portion of the first adhesive layer is configured to be in direct contact with at least portion of the tissue area of the patient when the device is applied to the patient.

25. The patient monitoring sensor of claim 14, wherein at least one surface of the conformable substrate body is configured to be in direct contact with a retention mechanism, and wherein the retention mechanism is configured to maintain the patient monitoring sensor in direct contact with the tissue area of the patient when applied to the patient.
